(19) United States
(12) Patent Application Publication
(10) Pub. No.: US 2025/0018048 A1
(43) Pub. Date: Jan. 16, 2025

(54) FLUCONAZOLE-COX INHIBITOR HYBRIDS: A DUAL-ACTING CLASS OF ANTIFUNGAL AZOLES

(71) Applicant: Ramot at Tel-Aviv University Ltd., Tel Aviv (IL)

(72) Inventors: MICHA FRIDMAN, Kiryat Bialik (IL); REBECA ELIAS, Lod (IL); PALLABITA BASU, Basirhat (IN)

(21) Appl. No.: 18/710,002

(22) PCT Filed: Nov. 16, 2022
(86) PCT No.: PCT/IB2022/061031

§ 371 Date: May 14, 2024

(51) Int. Cl. A61K 47/55; A01N 43/653; A01P 3/00; A61P 31/10

(52) U.S. Cl. CPC A61K 47/55; A01N 43/653; A01P 3/00; A61P 31/10

(57) ABSTRACT

A novel dual-acting class of antifungal compound synthesized by linking an azole pharmacophore to a COX inhibitor to form a hybrid molecule. A method of making the antifungal compound by preparing a mixture of an azide-functionalized pharmacophore, subjecting the azide-functionalized pharmacophore to catalytic hydrogenation to obtain an amine-functionalized azole pharmacophore, and coupling a COX inhibitor to the amine-functionalized azole pharmacophore. A method for treating fungal infection in plants and animals comprising administering the antifungal compound.

### BACKGROUND

Although humans and yeast have been evolving along different paths over a period of a billion years, there is still a significant resemblance between the genomes of human and both friendly and pathogenic yeast. Approximately one-third of the genes found in the human genome have counterparts in the genomes of yeast; amino acid sequences of the human proteome overlap by more than 30% with those of the yeast proteome. Moreover, when 414 human genes were inserted into yeast cells one at a time, approximately 50% of them were functional and facilitated the survival of the yeast cells. It is, therefore, no wonder that, compared to the relative abundance of unique drug targets in bacteria, very few such targets are suitable for selective inhibition in pathogenic yeast.

Prevention and treatment of fungal infections relies on a relatively limited number of antifungal drugs in only four major drug classes: azoles, echinocandins, allylamines, and polyenes. The incidence of fungal infections has risen sharply in recent decades due to rising numbers of susceptible immunosuppressed persons and higher prevalence of intrinsically drug-resistant and drug-tolerant species. Global epidemics are increasingly being caused by drug resistant (and multi-drug resistant) fungal pathogens including Aspergillus fumigatus, Candida glabrata, Cryptococcus neoformans, and, recently, Candida auris, a pathogen with the potential for extensive multi-drug resistance. Notably, infections with drug-resistant fungi are associated with mortality rates in the range of 50%, granting them high priority for new drug development. An increasingly favored approach to overcome the shortage in fungal drug targets and drug classes is to expand and/or enhance the efficacy of existing antifungal drugs through combination therapies.

It was previously established that when used in combination nonsteroidal cyclooxygenase (COX) inhibiting anti-inflammatory drugs, such as ibuprofen, and azole antifungal drugs synergize to improve antifungal potency. Combination therapies can be affected by differences in pharmacological properties and by side effects of drugs used in the combination. To date, several FDA-approved drugs have been reported to synergize with antifungal drugs including inhibitors of Hsp90, calcineurin, TOR and PKC pathways, and drug efflux inhibitors.

Several clinically used nonsteroidal anti-inflammatory drugs that act by inhibiting COX enzymes, including ibuprofen, aspirin, and indomethacin, have been shown to possess moderate antifungal activity; the mechanism is unknown. When used in high concentrations in combination with the most commonly used antifungal azole drug fluconazole (FLC, FIG. 3A), COX inhibitors significantly improve antifungal efficacy in vitro. The antifungal efficacy of this combination was validated in animal models. Ibuprofen effectively synergizes with FLC against azole-resistant C. albicans. A similar effect was observed for a combination of FLC and FK506, a 23-membered-ring macrolide immunosuppressant that also acts as a broad-spectrum inhibitor of pleiotropic drug resistance ATP-binding cassette transporters. FLC-resistant isolates revert to FLC susceptible after incubation with ibuprofen yet retain high levels of expression of CDR1 and CDR2 efflux pumps. It was shown that ibuprofen can alter the expression of the genes encoding the efflux pumps and that it may act directly as an efflux pump blocker.

The arachidonic acid pathway has been associated with the yeast-to-hyphae morphogenesis in several species of the genus Candida, the most commonly diagnosed pathogen causing fungal-born infectious diseases in humans. In mammals, nonsteroidal anti-inflammatory drugs such as COX inhibitors reduce the formation of prostaglandins generated via the arachidonic acid pathway. Prostaglandins are involved in the morphogenesis and pathogenicity of yeast and mediate the host inflammatory response. Prostaglandin E2 (PGE2) regulates growth and colonization and promotes the formation of biofilms of several Candida species. Several studies have shown that reduced PGE2 production limits the virulence of pathogenic fungi suggesting that the use of inhibitors of the arachidonic acid pathway could improve outcomes of fungal infections.

Physicians are reluctant to prescribe COX inhibitors to patients suffering from infections due to their anti-inflammatory effects as these agents reduce the ability of the innate immune system to combat the pathogen. The efficacy of combination treatments heavily relies on the pharmacokinetic and pharmacodynamic properties of each of the drugs in the combination. Moreover, COX-inhibiting drugs are known to induce gastropathy that can result in internal bleeding and digestive system ulcers. This side effect has been attributed to the carboxylic acid functionality that is common to all classical COX-inhibiting nonsteroidal anti-inflammatory drugs. Ester and amide derivatives of these drugs maintain COX inhibition but can cause less gastropathy.

To overcome the clinical shortcomings of presently available COX inhibitors, the present disclosure relates to a novel type of antifungal synthesized by linking an azole pharmacophore with a COX inhibitor to form a hybrid drug molecule with increased antifungal properties and decreased COX inhibitor-associated negative side effects such as gastropathy. The hybrid molecule incorporates the antifungal properties of COX inhibitors with those of antifungal azoles by conjugating the amine-functionalized pharmacophore of FLC to different COX inhibitors via their carboxylic acid to form hybrid drugs.

### SUMMARY

By overcoming limitations of separately administering combinations of azole antifungals and COX inhibitors, this disclosure relates to a novel type of antifungal synthesized by linking an azole pharmacophore to a COX inhibitor to form a hybrid drug molecule. These hybrid drug molecules were prepared by conjugation of a chiral azole pharmacophore with a COX inhibitor which, in certain embodiments, can be selected from a collection of chiral and achiral COX inhibitors to form an azole pharmacophore-COX inhibitor hybrid drug molecule.

In one aspect, this disclosure relates to a novel anti-fungal hybrid of an azole pharmacophore linked to an Ru, wherein Ru=a COX Inhibitor. In certain embodiments, Ru may be linked to the Azole pharmacophore via a link, wherein the link is formed by a direct chemical bond, a linker moiety, or a chemically bonded linker moiety.

Another aspect of the disclosure relates to a method for the treatment of fungal infections. In one embodiment, the azole pharmacophore-COX inhibitor hybrid drug molecule can be designed and optimized for optimal efficacy depending on the intended use, such as for use as an antifungal in medicine or agriculture. For example, certain embodiments of the molecule may be more efficacious as an antifungal agent in plant models. Other embodiments may be more efficacious as an antifungal agent in animal models. In some embodiments, the antifungal can be a plastic or polymer additive.

The present disclosure further relates to a process for preparing the antifungal compounds, pharmaceutical and agricultural preparations containing the antifungal compounds for prevention and treatment of fungal infections, as well as methods for treating said infections. Additional embodiments and features are described in the detailed description which follows.

### BRIEF DESCRIPTION OF THE DRAWINGS

FIG. 1. Chemical structures of synthesized COX-inhibitor-azole hybrids with numbered protons.

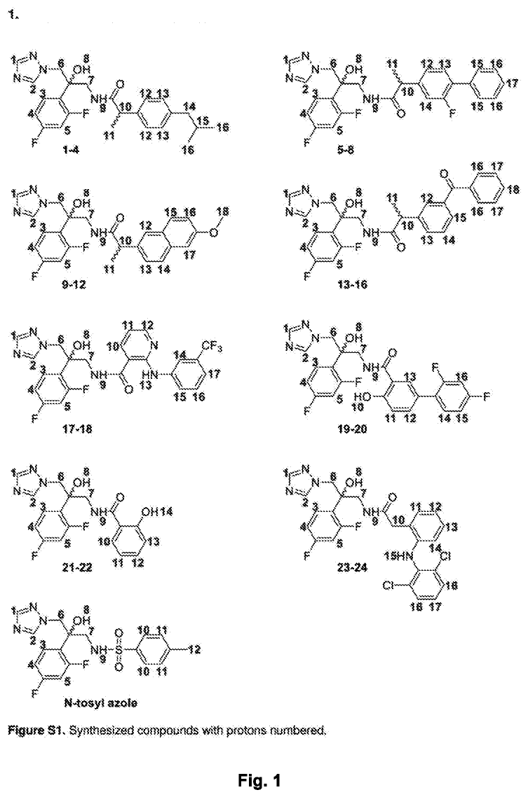

FIG. 2. General synthesis of diastereomers and enantiomers of fluconazole-COX inhibitor hybrids.

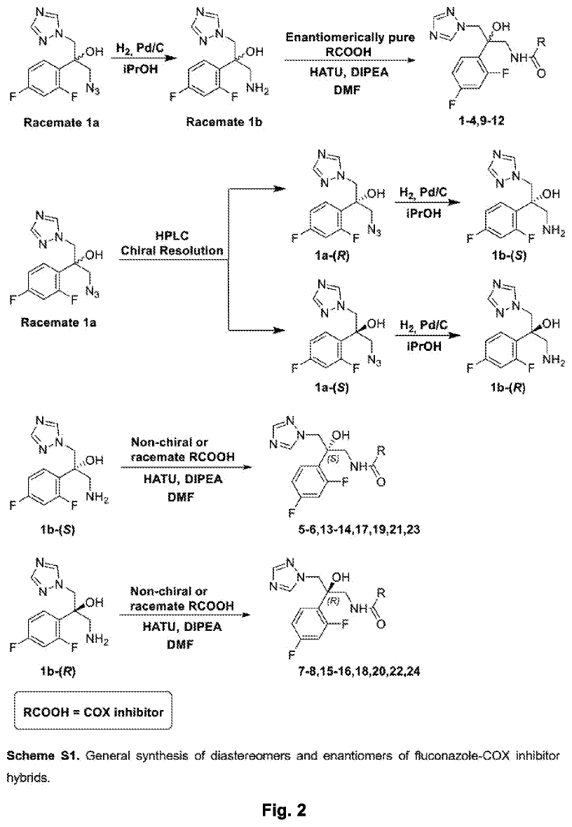

FIG. 3. (A) Structure of clinically used antifungal azole drugs fluconazole and voriconazole. (B) Synthesis of enantiomerically pure azole pharmacophores. (C) Synthesis of crystallizable N-tosyl derivatives of the enantiomerically pure pharmacophores and X-ray structures confirming their absolute configuration.

FIG. 4. Chemical structures of synthesized COX-inhibitor-azole hybrids.

FIG. 5. Antifungal activities (MIC values) of clinically used FLC and VOR and of the three most potent azole-COX inhibitor hybrids 1, 2, and 5.

FIG. 6. The effect of chirality on antifungal activity against C. albicans strains. Black circles represent MIC₈₀ values of FLC and VOR.

FIG. 7. A comparison of antifungal tolerance as measured by disk diffusion assays for FLC, VOR, and azole-COX inhibitor hybrids 1 and 5.

FIG. 8. The effect of azole-COX inhibitor hybrids 1 and 5 on the growth of C. albicans lacking CYP51, the target of antifungal azoles.

FIG. 9. Table S5 which provides the MIC values of hybrids 1-24 against C. glabrata.

### DETAILED DESCRIPTION OF EMBODIMENTS

As described herein, the present disclosure relates to an antifungal compound described in detail in connection with certain preferred and optional embodiments so that various aspects thereof may be more fully understood and appreciated. Accordingly, the present disclosure provides a novel antifungal compound in the form of an azole pharmacophore-Cox inhibitor hybrid. In some embodiments, the hybrid can be according to Formula I, an (R) enantiomer thereof, an (S) enantiomer thereof, a diastereomer thereof, a racemate thereof, or a pharmaceutically or agriculturally acceptable salt thereof.

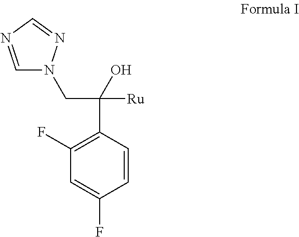

The antifungal compound comprises an azole pharmacophore linked to an Ru moiety via a link, wherein Ru is a COX inhibitor. The compound of Formula I can be obtained by conjugation of a chiral antifungal azole pharmacophore to a COX inhibitor. The COX inhibitor of the antifungal compound may be either chiral or achiral. In various embodiments, the link between the azole pharmacophore and COX inhibitor may be formed by a direct chemical bond, a linker moiety or molecule, or a chemically bound linker moiety or molecule.

The present disclosure also relates to a method of making the antifungal compound of Formula I, comprising preparing a racemic mixture of an azide-functionalized pharmacophore. The azide-functionalized pharmacophore is subjected to a catalytic hydrogenation step to obtain an amine-functionalized azole pharmacophore. The amine-functionalized azole pharmacophore is then coupled to a COX inhibitor to form the hybrid molecule. In some embodiments, the COX inhibitor is coupled via an amide bond between a primary amine of the amine-functionalized azole pharmacophore and a carboxylic acid of the COX inhibitor. In certain embodiments, the COX inhibitor is selected from the group consisting of ibuprofen, flurbiprofen, naproxen, ketoprofen, niflumic acid, diflunisal, salicylic acid, and diclofenac. It is envisioned that the COX inhibitor can be preferentially selected based on parameters of the COX inhibitor such as mechanism of action, adverse effects, and indications. This disclosure is not limited to any specific COX inhibitor or group or class of COX inhibitor.

In the present specification, the structural formula of the compound represents a certain isomer for convenience in some cases, but the present disclosure includes any and all isomers, atropic isomers, geometrical isomers, optical isomers based on an asymmetrical carbon, stereoisomers, diastereomers, tautomers, and the like, it being understood that not all isomers may have the same level of activity.

Throughout the description, where compositions are described as having, including, or comprising specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions can sometimes be changed so long as that which is being disclosed remains operable. Moreover, two or more steps or actions can sometimes be conducted simultaneously.

Non-steroidal anti-inflammatory COX inhibiting drugs can be selected from the following non-delimiting chiral and achiral drugs where appropriate: Salicylates, Aspirin (acetylsalicylic acid), Diflunisal (Dolobid), Salicylic acid and its salts, Salsalate (Disalcid), Propionic acid derivatives, Ibuprofen, Dexibuprofen, Naproxen, Fenoprofen, Ketoprofen, Dexketoprofen, Flurbiprofen, Oxaprozin, Loxoprofen, Pelubiprofen, Zaltoprofen, Acetic acid derivatives, Indomethacin, Tolmetin, Sulindac, Etodolac, Ketorolac, Diclofenac, Aceclofenac, Bromfenac, Nabumetone, Enolic acid (Oxicam) derivatives, Piroxicam, Meloxicam, Tenoxicam, Droxicam, Lornoxicam, Isoxicam, Phenylbutazone (Bute), Anthranilic acid derivatives (Fenamates), derivatives of fenamic acid and anthranilic acid and salicylic acid and aspirin, Meclofenamic acid, Flufenamic acid, Tolfenamic acid, Selective COX-2 inhibitors (Coxibs), Celecoxib, Rofecoxib, Valdecoxib, Parecoxib, Lumiracoxib, Etoricoxib, Firocoxib used in animals such as dogs and horses, Sulfonanilides, Nimesulide, Clonixin, Licofelone, 5-LOX/COX inhibitor, and H-harpagide in figwort or devil's claw. It is envisioned that the COX inhibitor can be preferentially selected based on parameters of the COX inhibitor such as mechanism of action, adverse effects, and indications. This disclosure is not limited to any specific COX inhibitor or group or class of COX inhibitor.

In some embodiments, non-limiting examples of azole antifungals can include clotrimazole, econazole, ketoconazole, miconazole, tioconazole, triazoles such as fluconazole and itraconazole, and second-generation triazoles, including voriconazole, posaconazole and ravuconazole, which can be more potent and more active against resistant pathogens.

In certain embodiments, Ru may be linked to the Azole pharmacophore via a link, wherein the link is formed by a chemical bond, a linker moiety, or a chemically bonded linker moiety. The chemical bond can include bonds such as an ionic bond, a covalent bond (e.g., carbon-carbon, carbon-oxygen, oxygen-silicon, sulfur-sulfur, phosphorus-nitrogen, carbon-nitrogen, metal-oxygen, or other covalent bonds), a hydrogen bond (e.g., between hydroxyl, amine, carboxyl, thiol, and/or similar functional groups), a dative bond (e.g., complexation or chelation between metal ions and monodentate or multidentate ligands), Van der Waals interactions, and the like. The linker encompasses a conjugating functionality suitable for attachment of the azole pharmacophore to the COX inhibitor via linker moieties. Linkers can generally be divided into cleavable and non-cleavable, can comprise a spacer unit, and generally, link two small molecules with defined molecular targets, such as azole pharmacophores and COX inhibitors, into one molecule.

Coupling reactions according to the disclosure such as, for example, for coupling a COX inhibitor to an azole pharmacophore wherein the COX inhibitor is coupled to the azole pharmacophore via an amide bond between a primary amine of the azole pharmacophore and a carboxylic acid of the COX inhibitor can be performed using coupling reagents suitable for said reaction. For example, in certain embodiments, where HATU-based amide bond formation is disclosed, it is also envisioned that other coupling reagents might also be suitable that are capable of forming a similar amide bond or coupling an azole pharmacophore and COX inhibitor to create a similarly efficacious hybrid pharmacophore-COX inhibitor compounds. Non-limiting coupling reagents include, for example, HATU, HBTU, TBTU, TATU, HCTU, Carbodiimides, DCC, DIC, EDC HCL, BOP, PYBOP, PyAOP, PyBROP, BOP-CL, COMU, TBTU, TSTU, TNTU, TPTU, TDBTU, TSTU, DEPBT, CDI, TCFH, etc.

In certain embodiments, infections of the following non-limiting examples of Candida species are contemplated for treatment with the compound and compositions of the disclosure: Candida albicans, Candida amphixiae, Candida antarctica, Candida argentea, Candida ascalaphidarum, Candida atlantica, Candida atmosphaerica, Candida auris, Candida blankii, Candida blattae, Candida bracarensis, Candida bromeliacearum, Candida carpophila, Candida carvajalis, Candida cerambycidarum, Candida chauliodes, Candida corydali, Candida dosseyi, Candida dubliniensis, Candida ergatensis, Candida fermentati, Candida fructus, Candida glabrata, Candida guilliermondii, Candida haemulonii, Candida humilis, Candida insectamens, Candida insectorum, Candida intermedia, Candida jeffresii, Candida kefyr, Candida keroseneae, Candida krusei, Candida lusitaniae, Candida lyxosophila, Candida maltosa, Candida marina, Candida membranifaciens, Candida mogii, Candida oleophila, Candida oregonensis, Candida parapsilosis, Candida quercitrusa, Candida rhizophoriensis, Candida rugosa, Candida sake, Candida sharkiensis, Candida shehatea, Candida sinolaborantium, Candida sojae, Candida stellata, Candida subhashii, Candida temnochilae, Candida tenuis, Candida theae, Candida theae, Candida tolerans, Candida tropicalis, Candida tsuchiyae, Candida ubatubensis, Candida viswanathii, Candida zemplinina.

In certain embodiments, non-limiting examples of infectious fungi that are contemplated for treatment with the combinatorial therapeutic compositions and methods described herein include, but are not limited to: Candida spp.; Cryptococcus spp.; Aspergillus spp.; Microsporum spp.; Trichophyton spp.; Epidermophyton spp.; Trichosporon spp.; Tinea versicolor; Tinea barbae; Tinea corporis; Tinea cruris; Tinea manuum; Tinea pedis; Tinea unguium; Tinea faciei; Tinea imbricate; Tinea incognito; Epidermophyton floccosum; Microsporum canis; Microsporum audouinii; Trichophyton interdigitale; Trichophyton mentagrophytes; Trichophyton tonsurans; Trichophyton schoenleini; Trichophyton rubrum; Hortaea werneckii; Piedraia hortae; Malasserzia furfur; Coccidioides immitis; Coccidioides posadasii; Histoplasma capsulatum; Histoplasma duboisii; Lacazia loboi; Paracoccidioides brasiliensis; Blastomyces dermatitidis; Sporothrix schenckii; Penicillium marneffei; Candida albicans; Candida glabrata; Candida tropicalis; Candida lusitaniae; Candida jirovecii; Exophiala jeanselmei; Fonsecaea pedrosoi; Fonsecasea compacta; Phialophora verrucosa; Geotrichum candidum; Pseudallescheria boydii; Rhizopus oryzae; Muco indicus; Absidia corymbifera; Synceplasastrum racemosum; Basidiobolus ranarum; Conidiobolus coronatus; Conidiobolus incongruous; Cryptococcus neoformans; Enterocytozoan bieneusi; Encephalitozoon intestinalis; and Rhinosporidium seeberi.

Many fungal animal and plant pathogens and diseases have been documented in the literature. In some embodiments, the compound can be used to treat these fungal pathogens and diseases. In some embodiments, the compound can be used to treat those fungal pathogens and diseases which are associated with the arachidonic acid pathway. In some embodiments, the compound can be used to treat those fungal pathogens and diseases which contain azole and/or COX-inhibitor-susceptible fungal strains.

As used herein, and unless otherwise specified, the terms “treat,” “treating” and “treatment” contemplate an action that occurs while a subject or surface is infected with fungal pathogens or disease or condition thereof, which reduces the potential for growth and spread or severity of the fungal pathogen infection, or retards or slows the progression of the infection or eliminates the same (“therapeutic treatment”), and also contemplates an action that occurs before a subject or surface becomes infected by fungal pathogens (“prophylactic treatment”). The term “treatment,” as used herein in the context of treating a fungal infection, pertains generally to treatment and therapy, whether of a human, animal (e.g., in veterinary applications), or plant (e.g., agricultural applications), in which some desired therapeutic effect is achieved.

In general, the “therapeutically effective amount” or “effective amount” of a compound refers to an amount sufficient to elicit the desired therapeutic or biological response such as the treatment of a fungal pathogen, or to delay, decrease, or eliminate infection or growth of a fungal pathogen. The effective amount of a compound may vary depending on such factors as the desired biological endpoint, the pharmacokinetics of the compound, the pathogen being treated, the mode of administration, and the age, health, and condition of the subject or conditions of a surface. An effective amount encompasses therapeutic and prophylactic treatment and includes the Minimum Inhibitory Concentration (MIC), a widely used measure of the susceptibility of yeasts to antifungal agents. The lowest concentration of antifungal drug that is sufficient to inhibit fungal growth is the MIC.

As used herein, and unless otherwise specified, a “prophylactically effective amount” of a compound is an amount sufficient to prevent establishment, growth, infection of a fungal pathogen or prevent its recurrence. A prophylactically effective amount of a compound means an amount of a therapeutic agent, in this case an azole-COX inhibitor hybrid drug, alone or in combination with other agents, which provides a prophylactic benefit in the prevention of infection or growth of a fungal pathogen. The term “prophylactically effective amount” can encompass an amount that improves overall prophylaxis or enhances the prophylactic efficacy of another prophylactic agent.

Also included are pharmaceutical compositions comprising one or more compounds as disclosed herein in an acceptable carrier, such as a stabilizer, buffer, and the like. The compositions can be formulated and used as sterile solutions and/or suspensions for injectable administration; lyophilized powders for reconstitution prior to injection/infusion; incorporated into polymers for various applications; topical compositions; as tablets, capsules, or elixirs for oral administration; or suppositories for rectal administration, and the other compositions known in the art.

The present disclosure also relates to a pharmaceutical composition comprising an antifungal compound of Formula I, in association with at least one pharmaceutically acceptable excipient/carrier. Additional embodiments envision an antifungal compound for use in plants comprising an antifungal compound of Formula I or an agriculturally acceptable salt thereof or the antifungal compound in association with an agriculturally acceptable carrier molecule.

The present disclosure also relates to a method for treating a fungal infection comprising administering a therapeutically effective amount of an antifungal compound. This disclosure should not be limited to the treatment of any one specific fungal genus or species where the compound is effective against many fungi. Certain aspects of this disclosure relate to treatment of a fungal infection by at least one fungus of the genus Candida. For example, the antifungal compound would be effective against infection by at least one fungus selected from the group consisting of C. albicans, C. glabrata, C. parapsilosis, C. tropicalis, C. guilliermondii, C. dubliniensis, and C. auris. In certain embodiments, it is envisioned that the antifungal compound has antifungal properties corresponding to an MIC (μg/mL) value ranging from approximately 0.003-10, from 0.003-4, from 0.003-2, from 0.003-1, from 0.003-0.5, from 0.003-0.25, from 0.003-0.12, from 0.003-0.06, from 0.003-0.03, from 0.003-0.015, and from 0.003-0.007.

An evaluation of the antifungal activity in cell-based assays of 24 embodiments of the novel azole Pharmacophore and COX inhibitor drug molecule hybrid is described below. Hybrids derived from ibuprofen and flurbiprofen were one to two orders of magnitude more potent than the clinically used fluconazole and comparable to voriconazole against a panel of Candida pathogens. The potencies of hybrids composed of an S-configured azole pharmacophore were higher than the corresponding hybrids with the R-configured pharmacophore. Tolerance, defined as the ability of a subpopulation of cells to grow in the presence of the drug, to the azole-COX inhibitor hybrids was lower than to fluconazole and voriconazole. The hybrids were active against a mutant that lacks the cytochrome P450 CYP51, which is the target of azole drugs, indicating that these agents act via a dual mode of action. The evaluation established that azole-COX inhibitor hybrids are potent antifungals with a dual mode of action. These agents are a novel class of antifungals with clinical potential.

The antifungal activity profiles of the hybrids were tested against a diverse panel of Candida representing several species of this common fungal pathogen and compared to activities of the clinically used azole drugs FLC and VOR. The antifungal activities of several hybrids were superior to that of FLC. Two hybrids, ibuprofen-based 1 and flurbiprofen-based 5, stood out due to potency significantly higher than FLC and comparable to VOR. Structure-activity relationship analysis revealed that all hybrids with an S-configured azole pharmacophore were more potent antifungals than the corresponding hybrids with an R-configured azole pharmacophore. No such generalization could be made for the chiral COX inhibitors. In all hybrids with a chiral COX inhibitor, the contribution of the chiral center of the azole pharmacophore to the antifungal activity of the hybrids was markedly higher compared to that of the chiral center of the COX inhibitor.

Importantly, analysis of tolerance, defined as the ability of a subpopulation of cells to grow in the presence of the drug, revealed that yeast cultures were less likely to be tolerant in the presence of the hybrids 1 and 5 than in the presence of FLC and VOR. Clinical isolates with high tolerance are associated with persistent candidemia, suggesting that lower levels of tolerance to a drug may reduce the chances of the persistence and/or reoccurrence of the infection.

Mechanistic investigation revealed that unlike the clinically used FLC and VOR that target CYP51 as their main mode of action, hybrids 1 and 5 retained activity against an erg11ΔΔ/erg3ΔΔ mutant C. albicans strain, which lacks CYP51. This activity was significantly lower, however, than the activity of these hybrids against the parent C. albicans strain from which the mutant lacking the target was derived. This indicates that the antifungal activity of the hybrids results mainly from the inhibition of CYP51 yet, unlike FLC and VOR, the hybrids also act via a second mode of action contributed by the COX-inhibiting segment.

This disclosure relates to the development of a novel type of potent antifungal agent that incorporates the antifungal activities of azoles and COX inhibitors in a single hybrid molecule. These new antifungals have impressive antifungal potency and, importantly, reduced levels of tolerance. The molecular hybrids described herein, based on FLC conjugated to ibuprofen or to flurbiprofen, serve as a novel alternative to currently available antifungal treatments.

### Synthetic Procedures

### Racemates 1a, 1b

N-tosyl azole (S). 1b-(R) (40 mg, 0.16 mmol) was dissolved in dry pyridine (2 mL) under argon at 0° C. then treated with tosyl chloride (39 mg, 0.20 mmol) and stirred at 0° C. The reaction was monitored by TLC (petroleum ether/ethyl acetate, 1:1). Upon completion at 2 h, the product was extracted with ethyl acetate, washed with H2O, dried over MgSO4, and concentrated to give the crude enantiomer. The concentrated crude was purified by preparative RP-HPLC to afford the compound N-tosyl azole (S) (51 mg, 80%). 1H NMR (400 MHz, CD₃OD) δ 8.30 (s, H-2, 1H), 7.74 (s, H-1, 1H), 7.66 (d, J=8.5, H-10, 2H), 7.43-7.32 (m, H-3, H-11, 3H), 6.86-6.78 (m, H-4, H-5, 2H), 4.72 (d, J=14.4 Hz, H-6, 1H), 4.64 (d, J=14.4 Hz, H-6, 1H), 3.38 (d, J=13.7 Hz, H-7, 1H), 3.33 (d, J=13.7 Hz, H-7, 1H), 2.42 (s, H-12, 3H). 13C NMR (100 MHz, CD₃OD) δ 164.5 (dd, 1JC-F=247.7 Hz, 3JC-F=13.2 Hz), 160.8 (dd, 1JC-F=246.0 Hz, 3JC-F=11.6 Hz), 151.5, 146.3, 145.0, 138.9, 131.6, 131.0, 128.2, 125.1, 112.2, 105.2, 75.8, 56.7, 50.7, 21.6. 19F NMR (375 MHz, CD₃OD) δ+−109.30 (m, Fpara), −113.00 (m, Fortho).

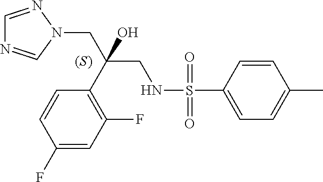

N-tosyl azole (R). 1b-(S) (20 mg, 0.08 mmol) was dissolved in dry pyridine (1 mL) under argon at 0° C. then treated with tosyl chloride (20 mg, 0.10 mmol) and stirred at 0° C. The reaction was monitored by TLC (petroleum ether/ethyl acetate, 1:1). Upon completion at 2 h, the product was extracted with ethyl acetate, washed with H2O, dried over MgSO4, and concentrated to give the crude enantiomer. The concentrated crude was purified by preparative RP-HPLC to afford the compound N-tosyl azole (R) (26 mg, 81%). 1H NMR (400 MHz, CD₃OD) δ 8.39 (s, H-2, 1H), 7.80 (s, H-1, 1H), 7.66 (d, J=8.1, H-10, 2H), 7.43-7.34 (m, H-3, H-11, 3H), 6.88-6.80 (m, H-4, H-5, 2H), 4.72 (d, J=14.6 Hz, H-6, 1H), 4.64 (d, J=14.6 Hz, H-6, 1H), 3.38 (d, J=13.6 Hz, H-7, 1H), 3.33 (d, J=13.6 Hz, H-7, 1H), 2.43 (s, H-12, 3H). 13C NMR (100 MHz, CD₃OD) δ 164.5 (dd, 1JC-F=248.6 Hz, 3JC-F=12.52 Hz), 160.7 (dd, 1JC-F=247.0 Hz, 3JC-F=12.5 Hz), 151.1, 146.1, 145.0, 138.9, 131.6, 130.9, 128.2, 125.1, 112.2, 105.2, 75.7, 56.8, 50.7, 21.6. 19F NMR (375 MHz, CD₃OD) δ+−109.34 (m, Fpara), −113.04 (m, Fortho).

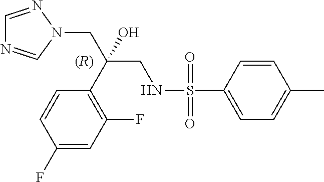

### Synthesis of Diastereoisomers and Enantiomers of Azole-COX Inhibitor Hybrids

To synthesize the hybrids composed of an azole and a COX inhibitor, first a racemic mixture of 1a, the azide-functionalized pharmacophore of the first- and second-generation antifungal azole drugs FLC and voriconazole (VOR) (Scheme 1A), was prepared. Enantiomerically pure 1a-(S) and 1a-(R) were readily obtained by HPLC using a preparative amylose-based chiral resolution column.

The azide-functionalized pharmacophores were then subjected to catalytic hydrogenation to afford the corresponding amine-functionalized derivatives 1b-(S) and 1b-(R), which were used for the conjugation of the COX-inhibitors via HATU-based amide bond formation. The absolute configurations of the two enantiomers of the azole pharmacophore were assigned by solving the X-ray structures of crystals of the two enantiomerically pure N-tosyl derivatives of the amine-functionalized derivatives 1b-(S) and 1b-(R), which readily crystalized from acetonitrile (Scheme 1C).

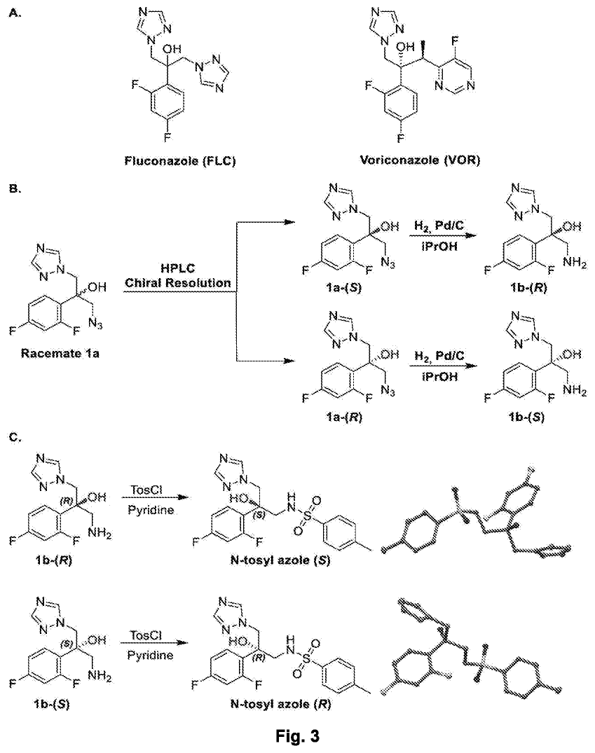

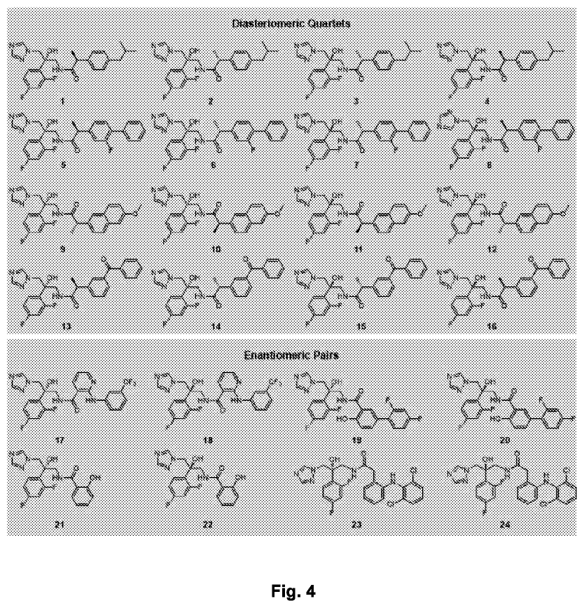

FIG. 3(A) shows the structure of clinically used antifungal azole drugs fluconazole and voriconazole. FIG. 3(B) shows the synthesis of enantiomerically pure azole pharmacophores. FIG. 3(C) shows the synthesis of crystallizable N-tosyl derivatives of the enantiomerically pure pharmacophores and X-ray structures confirming their absolute configuration. FIG. 4 shows the chemical embodiments of synthesized COX-inhibitor-azole hybrids.

Next, the hybrid antifungal molecules were synthesized by coupling COX inhibitors to each of the two azole enantiomers by forming an amide bond between the primary amine of the azole pharmacophore and the carboxylic acid of the COX inhibitor. Four of the COX inhibitors, ibuprofen, flurbiprofen, naproxen, and ketoprofen, contain a chiral center and were used for the generation of all four diastereomers of each hybrid (1-4, 5-8, 9-12 and 13-16, respectively, FIG. 4). The achiral COX inhibitors niflumic acid, diflunisal, salicylic acid, and diclofenac were used in the synthesis of enantiomeric azole pairs (17-24, respectively, FIG. 4). The purities of the 24 hybrids were determined by chiral semi-preparative HPLC column and confirmed to be >95% (Table S1, Figures S2-S25). The structures of the hybrids synthesized were verified using ¹H, ¹³C, and ¹⁹F NMR (Figures S28-S99) and HRMS.

TABLE S1

| | Time | Purity | ee |
|---|---|---|---|
| Compound | (min) | (%) | (%) |
| 1 | 10.03 | >99 | >99 |
| 2 | 8.25 | >99 | 98 |
| 3 | 7.57 | >99 | >99 |
| 4 | 7.10 | >99 | >99 |
| 5 | 7.30 | >99 | >99 |
| 6 | 7.40 | >99 | >99 |
| 7 | 8.65 | >99 | 98 |
| 8 | 7.98 | >99 | >99 |
| 9 | 6.93 | >99 | >99 |
| 10 | 6.53 | >99 | >99 |
| 11 | 7.50 | >99 | >99 |
| 12 | 6.83 | >99 | >99 |
| 13 | 5.57 | >99 | >99 |
| 14 | 5.62 | >99 | >99 |
| 15 | 8.52 | >99 | >99 |
| 16 | 6.25 | >99 | >99 |
| 17 | 5.78 | >99 | 98 |
| 18 | 6.17 | >99 | >99 |
| 19 | 6.08 | >99 | >99 |
| 20 | 7.85 | >99 | >99 |
| 21 | 3.93 | 96 | >99 |
| 22 | 4.15 | 96 | 95 |
| 23 | 6.60 | >99 | >99 |
| 24 | 7.12 | >99 | >99 |
| N-tosyl azole (S) | 4.15 | >99 | >99 |
| N-tosyl azole (R) | 5.60 | 98 | 97 |

Table S1 shows HPLC conditions for separation. For compounds 1-18, 21-24, N-tosyl azole (S), and N-tosyl azole (R), Solvent A was 40% H₂O and solvent B was 60% Acetonitrile. For compounds 19-20, Solvent A was 0.1% TFA in 40% H₂O and solvent B was 60% acetonitrile. The flow rate was 5 ml/min.

### Antifungal Potencies of the Hybrids and the Effects of Chiral Centers.

The antifungal activities of the 24 azole-COX inhibitor hybrids were evaluated against a panel of 16 representative strains of different species of the genus Candida. Candida species cause both superficial and systemic infections. The panel included strains of C. albicans, C. glabrata, C. parapsilosis, C. tropicalis, C. guilliermondii, C. dubliniensis, and C. auris (Table S2). To evaluate antifungal activity, the minimal inhibitory concentration 80% (MIC₈₀) values were determined, which were defined as the lowest drug concentrations with turbidity (measured at OD₆₀₀) less than or equal to that of specific 1:5 dilutions of the growth control. As controls, FLC and VOR, were tested.

TABLE S2

| | | | Isogenic | | |
|---|---|---|---|---|---|
| # | Species | Strain Name | parental strain | Genotype | Source |
| 1. | C. albicans | SC5314 | WT | | David Perlin² |
| 2. | C. albicans | ATCC 90028 | — | | ATCC |
| 3. | C. albicans | ATCC 24433 | — | | ATCC |
| 4. | C. albicans | P-87 | clinical isolate | | Matthew P. |
| | | | | | Hirakawa³ |
| | | | | leu2Δ/leu2Δ | |
| | | | | his1Δ/his1Δ | |
| 5. | C. albicans | SN152 | — | arg4Δ/arg4Δ | Susan Lindquist⁴ |
| | | | | URA3/ura3Δ::imm434 | |
| | | | | IRO1/iro1Δ::imm | |
| 6. | C. albicans | BV11/ erg3Δ/Δs/ | SN152 | erg3Δ::C.d.HIS1/ | Susan Lindquist⁴ |
| | | g11Δ/Δ | | erg3Δ:C.m.LEU2 | |
| | | | | erg11ΔC.d.ARG4/ | |
| | | | | erg11Δ/C.d.ARG4 | |
| 7. | C. glabrata | T-50 (BG2) | clinical isolate | | Brendan |
| | | | | | Cormack⁵ |
| 8. | C. glabrata | ATCC 66032 | — | | ATCC |
| 9. | C. glabrata | ATCC 2001 | — | | Cecile Fairhead⁶ |
| 10. | C. glabrata | T-191 (11-331) | clinical isolate | | Ronen Ben Ami⁷ |
| 11. | C. glabrata | T-195 (11-064) | clinical isolate | | Ronen Ben Ami⁷ |
| 12. | C: parapsilosis | ATCC 22019 | | | ATCC |
| 13. | C. guilliermondii | T-47 (B-3163) | WT | | Mike |
| | | | | | McEachern⁸ |
| 14. | C. tropicalis | 660 | WT | | P. T. Magee⁹ |
| 15. | C. dubliniensis | T-99 (Wu284) | | | Gary Moran¹⁰ |
| 16. | C. auris | ATCC MYA5001 | | | ATCC |
| 17. | C. auris | ATCC MYA5002 | | | ATCC |

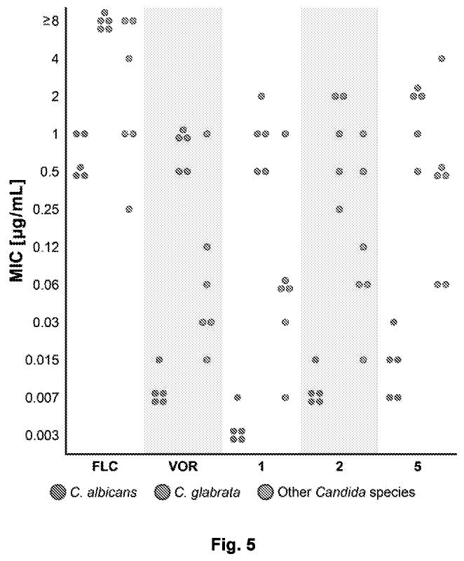

Table S2 provides yeast strain information. Of the 24 hybrids, three stood out as the most potent agents with the lowest MIC₈₀ values against all of the azole-susceptible strains in the panel: ibuprofen-based hybrids 1 and 2 and flurbiprofen-based hybrid 5 (FIG. 5 and Tables S3-S6). Of these three hybrids, ibuprofen-based azole 1 had the most potent activity against the majority of the azole-susceptible strains in the panel; this hybrid was up to two orders of magnitude more potent than FLC and was as potent as VOR.

FIG. 5 shows antifungal activities (MIC₈₀ values) of clinically used FLC and VOR and the three most potent azole-COX inhibitor hybrids 1, 2, and 5. MIC₈₀ values were determined using the broth microdilution method over a concentration range of 0.003-64 μg/mL. Orange circles represent C. albicans strains, yellow circles represent C. glabrata strains, and green circles represent C. parapsilosis, C. tropicalis, C. dubliniensis and C. auris. Cells were grown in YPAD medium at 30° C. (For C. auris strains 37° C.) 24 h. Each concentration was tested in triplicate, and results were confirmed in at least two independent experiments.

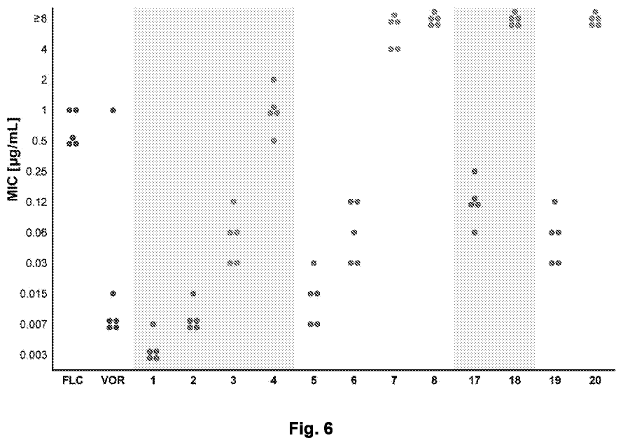

In search of structure-activity relationships, next the results of the antifungal activity tests were analyzed in the context of the chiral center or centers of the hybrids. Analysis revealed a clear connection between the absolute configuration of the chiral center at the carbon of the azole pharmacophore segment in both the diastereomeric tetrads and enantiomeric pairs. In all cases, the antifungal activity of hybrids with an S-configured carbon center of the azole pharmacophore segment had higher potency than the corresponding hybrids with the R-configured carbon center. Selected examples of two tetrads (ibuprofen-based 1-4 and flurbiprofen-based 5-8) and of two enantiomeric pairs (niflumic acid-based 17, 18 and diflunisal-based 19, 20)—which demonstrated the superior activity of the S- vs. R-configured carbon of the azole pharmacophore—are presented in FIG. 6.

FIG. 6 shows the effect of chirality on antifungal activity against C. albicans strains. Black circles represent MIC₈₀ values of FLC and VOR. Blue circles represent MIC₈₀ values of hybrids composed of an S-configured azole-pharmacophore, and pink circles represent MICso values of hybrids composed of an R-configured azole-pharmacophore.

No general correlation could be made between antifungal potency and the chiral center of the COX-inhibitor segments of the diastereomeric tetrads; rather, the results depended on the specific COX-inhibitor. For example, hybrid 3 composed of R-configured ibuprofen was more potent than the corresponding S-configured ibuprofen hybrid 4. In the flubiprofen tetrad, however, the S-configured flurbiprofen hybrid 5 was more potent than the corresponding R-configured ibuprofen hybrid 6. Of note, the chiral center of the azole pharmacophore markedly affected the antifungal activity of the hybrids, and the contribution of the chiral center of the COX inhibitor suggests that the main target of these antifungals is CYP51, the target of the azole class of antifungals. The investigation of the antifungal activity indicated that hybrids prepared by conjugation of the carboxylic acid of COX inhibitors to the amine-functionalized pharmacophore of the azole drug FLC can have markedly improved antifungal activity compared to that of FLC and comparable to that of the potent second-generation azole VOR.

Candida Tolerance to Azole-COX Inhibitor Hybrids is Lower than to FLC and VOR.

The majority of treatment failures for patients with invasive candidiasis are caused by apparently susceptible isolates. For example, during a clinical trial on the treatment of invasive candidiasis, the drug anidulafungin, which belongs to the echinocandin class of antifungal drugs that act by inhibiting cell-wall formation, was significantly superior to FLC, although the vast majority of isolates were susceptible to both drugs.

Apparently, susceptible isolates resist antifungal drugs by exhibiting tolerance, defined as the ability of a subpopulation of cells to grow slowly at supra-MIC concentrations. Activation of tolerance mechanisms depends on stress response pathways. Tolerance is, therefore, mechanistically distinct from resistance that relies upon mechanisms that are constantly under alert and do not require activation by stress response signals.

Since the subpopulation exhibiting antifungal tolerance is usually characterized by slow growth, it becomes visually detectible after at least 48 h of growth in the presence of the drug, whereas resistance is generally evident after 24 h. Since the common practice in clinical microbiology labs is to measure growth at 24 h, strains with high levels of tolerance may mistakenly be classified as drug-susceptible even though there may be a large percentage of drug-tolerant subpopulation in the culture.

The level of tolerance varies between isolates presumably due to genetic differences, and even within a single genetic isolate, tolerance responses of individual cells may differ significantly. Tolerance is thus the result of physiological or epigenetic differences rather than genetic variation.

Clinical isolates that cause persistent infections and that fail to respond to a single course of FLC have higher intrinsic tolerance levels than those isolates that cause non-persistent infections that are cleared with a single FLC course. This suggests that measurement of tolerance may provide useful prognostic information and there is a need for development of drugs that are unaffected by tolerance.

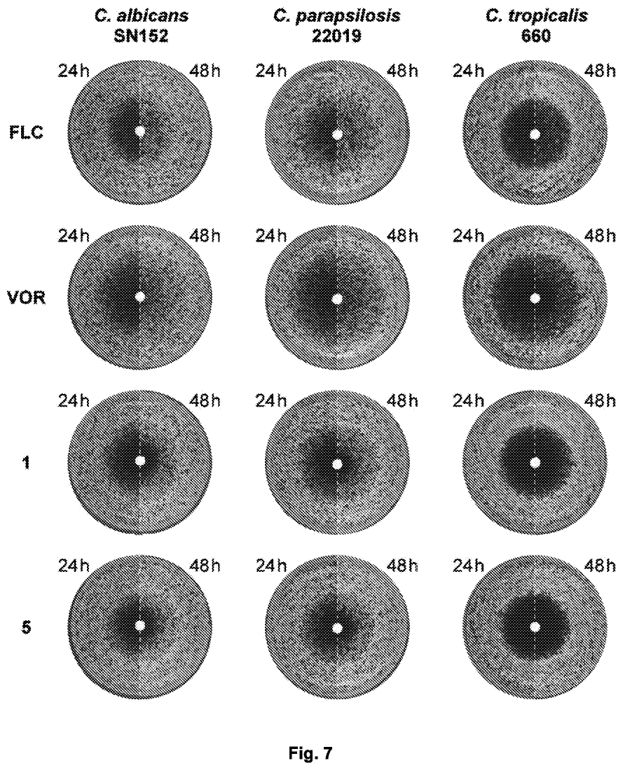

To investigate how tolerance is affected by the azole-COX inhibitor hybrids, a comparison of hybrids 1 and 5 to FLC and VOR in a disk diffusion assay was performed. Tolerance was evaluated by comparing the zone of inhibition after 24 h to that after 48 h. The assay was carried out on three representative strains: C. albicans, C. parapsilosis, and C. tropicalis (FIG. 7).

FIG. 7. shows a comparison of antifungal tolerance as measured by disk diffusion assays for FLC, VOR, and azole-COX inhibitor hybrids 1 and 5. As shown, compared to FLC and VOR, azole-COX inhibitor hybrids 1 and 5 display reduced antifungal tolerance. Disk diffusion assays were carried out on casitone agar plates containing disks loaded with 25 μg of the tested hybrids. Plates were imaged after 24 h to evaluate antifungal activity (left half of the plate image) and after 48 h to evaluate tolerance (right half of the plate image).

After 48 h of incubation with FLC or VOR disks, the zones of inhibition that had appeared after 24 h of incubation in plates seeded with C. albicans SN152 or with C. parapsilosis ATCC 22019 were covered by drug tolerant colonies; the drug tolerant subpopulation was smaller for C. tropicalis 660. All three tested strains displayed reduced tolerance to both hybrids 1 and 5 compared to the tolerance to FLC and VOR with the most pronounced effect observed in C. tropicalis 660 plates (FIG. 7). No correlation could be made between MIC₈₀ values and the level of tolerance.

Tolerance to compounds with lower or similar MIC values was not necessarily lower compared to tolerance to compounds with higher MIC₈₀ values. For example, the MIC₈₀ values of 5, and VOR against C. parapsilosis 22019 were 0.5 μg/mL, and 0.015 μg/mL, respectively (Tables S6), whereas the tolerance of this strain to hybrid 5 was markedly lower than that to VOR (FIG. 7). Since VOR acts predominantly by inhibition of CYP51, this suggests that the reduced tolerance to the azole-COX inhibitor hybrids is not exclusively due to inhibition of CYP51 and that the antifungal effect of their COX inhibitor segment is likely responsible for the observed lower tolerance to these agents.

TABLE S6

| | C. | C. | C. | C. |
|---|---|---|---|---|
| | parapsilosis | guilliermondii | tropicalis | dubliniensis |
| Compound | 22019 | T-47 | 660 | T-99 |
| FLC | 1 | 4 | 1 | 0.25 |
| VOR | 0.015 | 0.06 | 0.12 | 0.03 |
| 1 | 0.007 | 0.06 | 0.03 | 0.06 |
| 2 | 0.015 | 0.06 | 0.5 | 0.12 |
| 3 | 0.5 | 1 | 1 | 1 |
| 4 | 8 | 16 | >64 | 0.03 |
| 5 | 0,5 | 0.5 | 0.06 | 0.5 |
| 6 | 2 | 2 | 8 | 2 |
| 7 | 8 | 32 | 64 | 0.5 |
| 8 | 64 | 64 | >64 | 2 |
| 9 | 1 | 2 | 2 | 4 |
| 10 | 1 | 1 | 4 | 2 |
| 11 | 4 | 8 | 8 | 8 |
| 12 | 4 | 32 | >64 | 32 |
| 13 | 8 | 32 | 16 | 64 |
| 14 | 8 | 16 | 16 | 4 |
| 15 | >64 | >64 | >64 | 16 |
| 16 | >64 | >64 | >64 | 16 |
| 17 | 0.5 | 2 | 2 | 2 |
| 18 | 8 | >64 | >64 | 16 |
| 19 | 0.06 | 2 | 0.12 | 2 |
| 20 | 8 | >64 | >64 | 0.5 |
| 21 | 1 | 1 | 0.12 | 2 |
| 22 | 8 | 16 | 32 | >64 |
| 23 | 0.5 | 8 | >64 | 4 |
| 24 | >64 | >64 | >64 | 2 |

Table S6 shows the MIC₈₀ values of hybrids 1-24 against C. parapsilosis, C. guilliermondii, C. tropicalis, and C. dubliniensis. Cells were grown in YPAD medium at 30° C. for 24 h. Values are given in μg/mL.

### Azole-COX Inhibitor Hybrids Act Predominantly by Inhibiting of Ergosterol Biosynthesis.

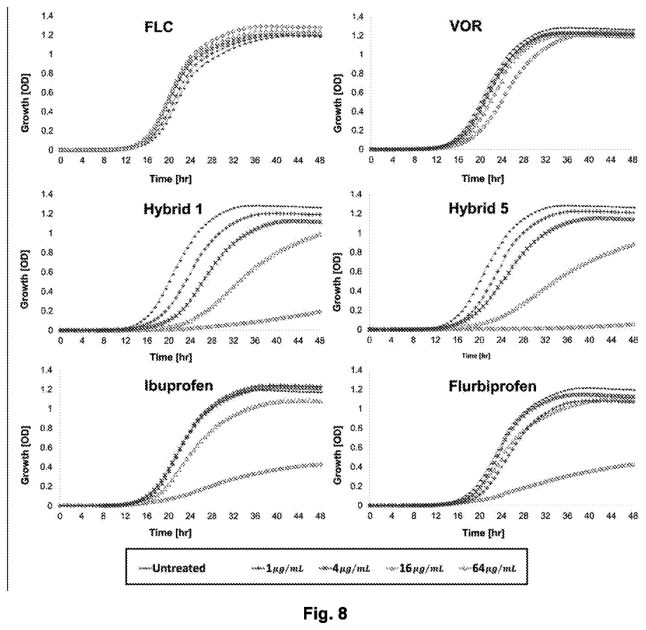

The clinically used FLC and VOR act primarily by preventing ergosterol biosynthesis via inhibition of CYP51. Analysis was done to determine whether fungal growth inhibition by the hybrids required the presence of the ERG11 gene that encodes for CYP51. The antifungal activities of hybrids 1 and 5 and of FLC and VOR were determined against an erg11ΔΔ/erg3ΔΔ mutant C. albicans strain and against C. albicans SN152 from which this double knockout strain was derived (Table S2). The erg11ΔΔ/erg3ΔΔ mutant is viable despite lacking CYP51 which is essential for aerobic growth, unless ERG3, which encodes a C-5 sterol desaturase, is inactive. Yeast growth was followed at OD₆₀₀ over 48 h in 96-well plates containing serial double dilutions of the tested hybrids. The results are summarized in FIG. 8.

FIG. 8. shows the effect of azole-COX inhibitor hybrids 1 and 5 on the growth of C. albicans lacking CYP51, the target of antifungal azoles. Cells of erg11ΔΔ/erg3ΔΔ mutant C. albicans were grown in YPAD media at 30° C. and treated with different concentrations of the tested hybrids. Growth was measured by recording the OD₆₀₀ values every 40 minutes over a 48-h course on an automated plate reader.

When CYP51 was not present, no significant effect on the growth of the double knockout mutant was observed for the entire range of concentrations of FLC. Modest reduction in growth was observed in wells treated with VOR at 64 μg/mL, the highest concentration tested, presumably due to non-specific effects of the drug at this high concentration. In contrast, a clear dose-dependent reduction in growth was evident in wells containing hybrids 1 or 5. Dose-dependent growth reduction was also observed in the presence of free ibuprofen and flurbiprofen, from which hybrids 1 and 5, respectively, were derived. Analysis suggests that the CYP51-independent antifungal effect of the novel azole-COX inhibitor hybrids 1 and 5 results from their COX inhibitor segments.

Of note, the MIC₈₀ values of hybrids 1 and 5 against the erg11ΔΔ/erg3ΔΔ mutant C. albicans strain were 64 μg/mL while FLC and VOR were inactive (Table S4). The MIC₈₀ values of these hybrids against the C. albicans SN152, the parent strain of the erg11ΔΔ/erg3ΔΔ mutant were 0.003 μg/mL and 0.007 μg/mL, respectively (Table S4). The high MICso values against the erg11ΔΔ/erg3ΔΔ mutant relative to those against the parent strain suggest that the contribution to the antifungal activity of the COX-inhibiting segment in these agents is modest compared to that of the inhibition of CYP51.

TABLE S4

| | C. albicans | C. albicans | C. albicans | C. albicans | C. albicans | C. albicans |
|---|---|---|---|---|---|---|
| Compound | SC5314 | 90028 | 24433 | P-87 | SN152 | 742 |
| FLC | 0.5 | 0.5 | 1 | 1 | 0.5 | >64 |
| VOR | 0.007 | 0.007 | 0.015 | 0.007 | 0.007 | >64 |
| 1 | 0.003 | 0.003 | 0.007 | 0.003 | 0.003 | 64 |
| 2 | 0.007 | 0.007 | 0.015 | 0.007 | 0.007 | 64 |
| 3 | 0.03 | 0.06 | 0.12 | 0.03 | 0.06 | 64 |
| 4 | 1 | 1 | 2 | 0.5 | 1 | >64 |
| 5 | 0.007 | 0.015 | 0.03 | 0.015 | 0.007 | 64 |
| 6 | 0.03 | 0.12 | 0.12 | 0.03 | 0.06 | >64 |
| 7 | 4 | 8 | 16 | 4 | 8 | 64 |
| 8 | 32 | 64 | >64 | 64 | 64 | >64 |
| 9 | 0.25 | 0.25 | 0.5 | 0.25 | 0.012 | 64 |
| 10 | 0.12 | 0.12 | 0.25 | 0.25 | 0.012 | >64 |
| 11 | 0.5 | 1 | 1 | 1 | 0.5 | >64 |
| 12 | 2 | 4 | 4 | 4 | 2 | >64 |
| 13 | 2 | 2 | 4 | 4 | 2 | >64 |
| 14 | 1 | 1 | 1 | 1 | 0.5 | >64 |
| 15 | >64 | >64 | >64 | >64 | >64 | >64 |
| 16 | >64 | >64 | >64 | >64 | >64 | >64 |
| 17 | 0.12 | 0.12 | 0.12 | 0.25 | 0.06 | 16 |
| 18 | 16 | 16 | 8 | 16 | 8 | 64 |
| 19 | 0.06 | 0.12 | 0.06 | 0.03 | 0.03 | 64 |
| 20 | 8 | 16 | 16 | 8 | 8 | 32 |
| 21 | 0.12 | 0.25 | 0.12 | 0.12 | 0.12 | >64 |
| 22 | 8 | 8 | 16 | 16 | 8 | >64 |
| 23 | 1 | 1 | 1 | 1 | 1 | >64 |
| 24 | >64 | >64 | >64 | >64 | >64 | >64 |

Table S4 shows the MIC values of hybrids 1-24 against C. albicans. Cells were grown in YPAD medium at 30° C. for 24 h. Values are given in μg/mL.

### EXPERIMENTAL SECTION

### Chemistry

General methods and instrumentation: ¹H-NMR spectra (including one-dimensional total correlation spectroscopy (1D-TOCSY)) were recorded on BrukerAvance™ 400 or 500 MHz spectrometers, and chemical shifts (reported in ppm) were calibrated to CD₃OD (β=3.31). ¹³C-NMR spectra were recorded on BrukerAvance™ 400 or 500 MHz spectrometers at 100 or 125 MHz. ¹⁹F-NMR spectra were recorded on BrukerAvance™ 400 or 500 MHz spectrometers at 375 or 470 MHz. Multiplicities are reported using the following abbreviations: s=singlet, d=doublet, t=triplet, q=quartet, dd=doublet of doublets, m=multiplet. Coupling constants (J) are given in Hz. High-resolution electrospray ionization (HRESI) mass spectra were measured on a Waters Synapt instrument. Chemical reactions were monitored by thin-layer chromatography (TLC) (Merck, Silica gel 60 F₂₅₄). Visualization was achieved using a cerium molybdate stain (5 g (NH₄)₂Ce(NO₃)₆, 120 g (NH₄)₆Mo₇O₂₄·4H₂O, 80 mL H₂SO₄, 720 mL H₂O) or with UV lamp. All chemicals, unless otherwise stated, were obtained from commercial sources. The following abbreviations are used:

DCM, dichloromethane; DIPEA, N,N-diisopropylethylamine; DMF, N,N-dimethylformamide; DMSO, dimethyl sulfoxide; FLC, fluconazole; HATU, hexafluorophosphate azabenzotriazole tetramethyl uronium; MeOH, methanol; MTT, Methylthiazolyldiphenyl-tetrazolium bromide; PBS, phosphate-buffered saline; TFA, trifluoroacetic acid; VOR, voriconazole and YPAD, yeast extract peptone adenine dextrose. hybrids were purified using Geduran® Si 60 chromatography (Merck).

The preparative reverse-phase high-pressure liquid chromatography (RP-HPLC) system used was an ECOM system equipped with a 5-μm, C-18 Phenomenex Luna Axia column (250 mm×21.2 mm). The mobile phase was acetonitrile in H2O, and the gradient was from 10% to 90% acetonitrile. The flow rate was 20 mL/min. Chiral semi-preparative high-pressure liquid chromatography (HPLC) used was performed on an ECOM system equipped with a 5-μm i-Amylose-3 Phenomenex Lux column (250 mm×10 mm). The flow rate was 5 mL/min.

### Crystallographic Data

Deposition Numbers 2116277 and 2166299 incorporated by reference contain the supplementary crystallographic data for this paper. These data are provided free of charge by the joint Cambridge Crystallographic Data Centre.

Hybrids 1 and 4. S-Ibuprofen (95 mg, 0.46 mmol) was dissolved in dry DMF (2 mL) under argon at 0° C. then treated with HATU (280 mg, 0.74 mmol) and DIPEA (0.27 mL, 1.55 mmol) and stirred for 10 min at 0° C. To the reaction mixture, racemate 1b (103 mg, 0.41 mmol) was added, and the solution was stirred at room temperature. The reaction was monitored by TLC (MeOH/DCM, 1:9). Upon completion after 3 h, the product was extracted with ethyl acetate, washed with H₂O, dried over MgSO₄, and concentrated to give the crude diastereomers. The concentrated crude was purified by column chromatography on SiO₂ using a gradient of MeOH/DCM as eluent to afford the diastereomer mix. The diastereomers were separated by preparative RP-HPLC to afford hybrids 1 and 4.

Hybrid 1 (65 mg, 73%). HRESI-MS m/z calculated for C₂₄H₂₈F₂N₄O₂Na, 465.2078; found for [M+Na]⁺, 465.2074. ¹H NMR (500 MHz, CD₃OD) b 8.29 (s, H-2, 1H), 7.76 (s, H-1, 1H), 7.37-7.32 (m, H-3, 1H), 7.01 (s, H-12, H-13, 4H), 6.87-6.83 (m, H-5, 1H), 6.74-6.71 (m, H-4, 1H), 4.65 (d, J=14.3 Hz, H-6, 1H), 4.53 (d, J=14.3 Hz, H-6, 1H), 3.88 (d, J=14.3 Hz, H-7, 1H), 3.55-3.43 (m, H-7, H-10, 2H), 2.42 (d, J=7.2 Hz, H-14, 2H), 1.87-1.75 (m, H-15, 1H), 1.28 (d, J=7.1 Hz, H-11, 3H), 0.88 (d, J=7.4 Hz, H-16, 6H). ¹³C NMR (125 MHz, CD₃OD) δ 177.4, 162.8 (dd, ¹J_{C-F}=246.2 Hz, ³J_{C-F}=12.2 Hz), 159.3 (dd, ¹J_{C-F}=245.4 Hz, ³J_{C-F}=12.0 Hz), 149.9, 144.7, 140.10, 138.3, 130.0, 128.8, 126.6, 123.7, 110.5, 103.5, 75.3, 55.6, 46.3, 45.3, 44.6, 30.0, 21.3, 17.3. ¹⁹F NMR (470 MHz, CD₃OD) δ −109.20 (m, Fₚₐᵣₐ), −113.12 (m, Fₒᵣₜₕₒ).

Hybrid 4 (49 mg, 55%). HRESI-MS m/z calculated for C₂₄H₂₈F₂N₄O₂Na, 465.2078; found for [M+Na]⁺, 465.2067. ¹H NMR (500 MHz, CD₃OD) δ 8.28 (s, H-2, 1H), 7.76 (s, H-1, 1H), 7.32-7.27 (m, H-3, 1H), 7.00 (s, H-12, H-13, 4H), 6.85-6.80 (m, H-5, 1H), 6.71-6.66 (m, H-4, 1H), 4.57 (d, J=14.3 Hz, H-6, 1H), 4.45 (d, J=14.3 Hz, H-6, 1H), 3.72 (d, J=14.3 Hz, H-7, 1H), 3.64 (d, J=14.3 Hz, H-7, 1H), 3.50 (q, J=7.0 Hz, H-10, 1H), 2.42 (d, J=7.2 Hz, H-14, 2H), 1.86-1.74 (m, H-15, 1H), 1.28 (d, J=7.1 Hz, H-11, 3H), 0.86 (d, J=6.6 Hz, H-16, 6H). ¹³C NMR (125 MHz, CD₃OD) δ 177.8, 162.8 (dd, ¹J_{C-F}=247.5 Hz, ³J_{C-F}=12.2 Hz), 159.2 (dd, ¹J_{C-F}=246.6 Hz, ³J_{C-F}=12.1 Hz), 149.9, 144.8, 140.1, 138.5, 130.0, 128.8, 126.6, 123.8, 110.6, 103.4, 75.6, 55.6, 46.7, 45.2, 44.5, 30.0, 21.3, 17.0. ¹⁹F NMR (470 MHz, CD₃OD) δ −109.62 (m, Fₚₐᵣₐ), −113.16 (m, Fₒᵣₜₕₒ).

Hybrids 2 and 3. R-Ibuprofen (99 mg, 0.48 mmol) was dissolved in dry DMF (2 mL) under argon at 0° C. then treated with HATU (299 mg, 0.79 mmol) and DIPEA (0.27 mL, 1.55 mmol) and stirred for 10 min at 0° C. To the reaction mixture, racemate 1b (105 mg, 0.41 mmol) was added, and the solution was stirred at room temperature. The reaction was monitored by TLC (MeOH/DCM, 1:9). Upon completion at 3 h, the product was extracted with ethyl acetate, washed with H₂O, dried over MgSO₄, and concentrated to give the crude diastereomers. The concentrated crude was purified by column chromatography on SiO₂ using a gradient of MeOH/DCM as eluent to afford the diastereomer mix. The diastereomers were separated by preparative RP-HPLC to afford hybrids 2 and 3.

Hybrid 2 (60 mg, 66%). HRESI-MS m/z calculated for C₂₄H₂₉F₂N₄O₂, 443.2259; found for [M+H]⁺, 443.2258. ¹H NMR (500 MHz, CD₃OD) δ 8.28 (s, H-2, 1H), 7.76 (s, H-1, 1H), 7.32-7.27 (m, H-3, 1H), 7.00 (s, H-12, H-13, 4H), 6.85-6.80 (m, H-5, 1H), 6.71-6.67 (m, H-4, 1H), 4.57 (d, J=14.3 Hz, H-6, 1H), 4.45 (d, J=14.3 Hz, H-6, 1H), 3.72 (d, J=14.3, H-7, 1H), 3.64 (d, J=14.3, H-7, 1H), 3.50 (q, J=7.1 Hz, H-10, 1H), 2.42 (d, J=7.2 Hz, H-14, 2H), 1.86-1.74 (m, H-15, 1H), 1.28 (d, J=7.1 Hz, H-11, 3H), 0.86 (d, J=6.6, H-16, 6H). ¹³C NMR (125 MHz, CD₃OD) δ 177.8, 162.82 (dd, ¹J_{C-F}=247.5 Hz, ³J_{C-F}=12.2 Hz), 159.16 (dd, ¹J_{C-F}=246.5 Hz, ³J_{C-F}=12.0 Hz), 149.9, 144.8, 140.1, 138.5, 130.0, 128.9, 126.6, 123.8, 110.6, 103.4, 75.6, 55.6, 46.7, 45.2, 44.6, 30.0, 21.3, 17.0. ¹⁹F NMR (470 MHz, CD₃OD) δ+−109.65 (m, Fₚₐᵣₐ), −113.19 (m, Fₒᵣₜₕₒ).

Hybrid 3 (78 mg, 85%). HRESI-MS m/z calculated for C₂₄H₂₈F₂N₄O₂Na, 465.2078; found for [M+Na]⁺, 465.2083. ¹H NMR (500 MHz, CD₃OD) δ 8.29 (s, H-2, 1H), 7.77 (s, H-1, 1H), 7.37-7.32 (m, H-3, 1H), 7.02 (s, H-12, H-13, 4H), 6.88-6.83 (m, H-5, 1H), 6.75-6.69 (m, H-4, 1H), 4.65 (d, J=14.3 Hz, H-6, 1H), 4.53 (d, J=14.3 Hz, H-6, 1H), 3.89 (d, J=14.3, H-7, 1H), 3.52-3.47 (m, H-7, H-10, 2H), 2.42 (d, J=7.2 Hz, H-14, 2H), 1.85-1.77 (m, H-15, 1H), 1.29 (d, J=7.1 Hz, H-11, 3H), 0.88 (d, J=6.6, H-16, 6H). ¹³C NMR (125 MHz, CD₃OD) δ 177.4, 162.8 (dd, ¹J_{C-F}=246.1, ³J_{C-F}=12.2 Hz), 159.3 (dd, ¹J_{C-F}=245.4, ³J_{C-F}=12.0 Hz), 149.9, 144.7, 140.1, 138.3, 130.0, 128.8, 126.6, 123.7, 110.5, 103.5, 75.3, 55.6, 46.3, 45.4, 44.6, 30.0, 21.3, 17.3. ¹⁹F NMR (470 MHz, CD₃OD) δ −109.19 (m, Fₚₐᵣₐ), −113.14 (m, Fₒᵣₜₕₒ).

Hybrids 5 and 6. Flurbiprofen (127 mg, 0.52 mmol) was dissolved in dry DMF (2 mL) under argon at 0° C. then treated with HATU (311 mg, 0.82 mmol) and DIPEA (0.27 mL, 1.55 mmol) and stirred for 10 min at 0° C. To the reaction mixture, 1b-(S) (100 mg, 0.39 mmol) was added, and the solution was stirred at room temperature. The reaction was monitored by TLC (MeOH/DCM, 1:9). Upon completion at 3 h, the product was extracted with ethyl acetate, washed with H₂O, dried over MgSO₄, and concentrated to give the crude diastereomers. The concentrated crude was purified by column chromatography on SiO₂ using a gradient of MeOH/DCM as eluent to afford the diastereomer mix. The diastereomers were separated by preparative RP-HPLC to afford hybrids 5 and 6.

Hybrid 5 (81 mg, 86%). HRESI-MS m/z calculated for C₂₆H₂₃F₃N₄O₂Na, 503.1671; found for [M+Na]⁺, 503.1670. ¹H NMR (500 MHz, CD₃OD) δ 8.32 (s, H-2, 1H), 7.79 (s, H-1, 1H), 7.52-7.50 (m, H-15, 2H), 7.44-7.41 (m, H-16, 2H), 7.37-7.30 (m, H-3, H-13, H-17, 3H), 7.00 (dd, J=8.0 Hz, 1.7 Hz, H-12, 1H), 6.96 (dd, J=11.9 Hz, 1.6 Hz, H-14, 1H), 6.88-6.83 (m, H-5, 1H), 6.70-6.65 (m, H-4, 1H), 4.67 (d, J=14.4 Hz, H-6, 1H), 4.59 (d, J=14.4 Hz, H-6, 1H), 4.02 (d, J=14.8 Hz, H-7, 1H), 3.58 (q, J=7.1 Hz H-7, 1H), 3.46 (d, J=14.1 Hz, H-7, 1H), 1.33 (d, J=7.1 Hz, H-11, 3H). ¹³C NMR (100 MHz, CD₃OD) δ 176.1, 162.8 (dd, ¹J_{C-F}=248.4 Hz, ³J_{C-F}=12.5 Hz), 159.4 (d, ¹J_{C-F}=245.3 Hz), 159.3 (dd, ¹J_{C-F}=247.3 Hz, ³J_{C-F}=11.4 Hz), 150.0, 144.7, 142.8, 135.5, 130.3, 130.0, 128.5, 128.1, 127.4, 127.3, 123.5, 123.2, 114.4, 110.3, 103.4, 75.2, 55.6, 46.1, 45.1, 17.3. ¹⁹F NMR (470 MHz, CD₃OD) δ+−109.03 (m, Fₚₐᵣₐ), −112.82 (m, Fₒᵣₜₕₒ), −119.72 (m, Fₘₑₜₐ).

Hybrid 6 (63 mg, 67%). HRESI-MS m/z calculated for C₂₆H₂₃F₃N₄O₂Na, 503.1671; found for [M+Na]⁺, 503.1668. ¹H NMR (500 MHz, CD₃OD) δ 8.34 (s, H-2, 1H), 7.80 (s, H-1, 1H), 7.53-7.51 (m, H-15, 2H), 7.46-7.42 (m, H-16, 2H), 7.38-7.30 (m, H-3, H-13, H-17, 3H), 7.00 (dd, J=8.0 Hz, 1.8 Hz, H-12, 1H), 6.95 (dd, J=11.8 Hz, 1.6 Hz, H-14, 1H), 6.88-6.83 (m, H-5, 1H), 6.70-6.66 (m, H-4, 1H), 4.60 (d, J=14.2 Hz, H-6, 1H), 4.54 (d, J=14.2 Hz, H-6, 1H), 3.80 (d, J=14.2 Hz, H-7, 1H), 3.65-3.55 (m, H-7, H-10, 2H), 1.33 (d, J=7.0 Hz, H-11, 3H). ¹³C NMR (125 MHz, CD₃OD) δ 178.5, 164.4 (dd, ¹J_{C-F}=247.9 Hz, ³J_{C-F}=12.4 Hz), 161.0 (d, ¹J_{C-F}=246.9 Hz), 160.7 (dd, ¹J_{C-F}=246.9 Hz, ³J_{C-F}=12.4 Hz), 151.5, 146.4, 144.5, 137.0, 131.9, 131.6, 130.1, 129.7, 129.1, 128.9, 125.3, 124.7, 116.0, 112.1, 104.9, 77.2, 57.3, 48.4, 46.5, 18.5. ¹⁹F NMR (470 MHz, CD₃OD) δ −109.69 (m, Fₚₐᵣₐ), −112.86 (m, Fₒᵣₜₕₒ), −119.73 (m, Fₘₑₜₐ).

Hybrids 7 and 8. Flurbiprofen (149 mg, 0.61 mmol) was dissolved in dry DMF (2 mL) under argon at 0° C. then treated with HATU (387 mg, 1.02 mmol) and DIPEA (0.40 mL, 2.29 mmol) and stirred for 10 min at 0° C. To the reaction mixture, 1b-(R) (130 mg, 0.51 mmol) was added, and the solution was stirred at room temperature. The reaction was monitored by TLC (MeOH/DCM, 1:9). Upon completion at 3 h, the product was extracted with ethyl acetate, washed with H₂O, dried over MgSO₄, and concentrated to give the crude diastereomers. The concentrated crude was purified by column chromatography on SiO₂ using a gradient of MeOH/DCM as eluent to afford the diastereomer mix. The diastereomers were separated by preparative RP-HPLC to afford hybrids 7 and 8.

Hybrid 7 (110 mg, 90%). HRESI-MS m/z calculated for C₂₆H₂₃F₃N₄O₂Na, 503.1671; found for [M+Na]⁺, 503.1670. ¹H NMR (500 MHz, CD₃OD) δ 8.32 (s, H-2, 1H), 7.80 (s, H-1, 1H), 7.53-7.51 (m, H-15, 2H), 7.45-7.42 (m, H-16, 2H), 7.38-7.31 (m, H-3, H-13, H-17, 3H), 7.00 (dd, J=8.0 Hz, 1.6 Hz, H-12, 1H), 6.96 (dd, J=11.9 Hz, 1.6 Hz, H-14, 1H), 6.88-6.83 (m, H-5, 1H), 6.70-6.66 (m, H-4, 1H), 4.69 (d, J=14.2 Hz, H-6, 1H), 4.60 (d, J=14.2 Hz, H-6, 1H), 4.02 (d, J=14.2 Hz, H-7, 1H), 3.58 (q, J=7.1 Hz, H-7, 1H), 3.47 (d, J=14.2 Hz, H-7, 1H), 1.33 (d, J=7.1 Hz, H-11, 3H). ¹³C NMR (125 MHz, CD₃OD) δ 177.6, 164.3 (dd, J_{C-F}=247.9 Hz, ³J_{C-F}=12.4 Hz), 160.9 (d, ¹J_{C-F}=246.9 Hz), 160.8 (dd, ¹J_{C-F}=246.9 Hz, ³J_{C-F}=11.4 Hz), 151.5, 146.2, 144.3, 137.0, 131.7, 131.5, 130.0, 129.6, 128.9, 128.8, 125.1, 124.7, 115.9, 111.8, 104.9, 76.7, 57.1, 47.6, 46.6, 18.8. ¹⁹F NMR (470 MHz, CD₃OD) δ+−109.03 (m, Fₚₐᵣₐ), −112.86 (m, Fₒᵣₜₕₒ), −119.75 (m, Fₘₑₜₐ).

Hybrid 8 (107 mg, 87%). HRESI-MS m/z calculated for C₂₆H₂₃F₃N₄O₂Na, 503.1671; found for [M+Na]⁺, 503.16680. ¹H NMR (400 MHz, CD₃OD) δ 8.34 (s, H-2, 1H), 7.80 (s, H-1, 1H), 7.54-7.51 (m, H-15, 2H), 7.46-7.42 (m, H-16, 2H), 7.39-7.30 (m, H-3, H-13, H-17, 3H), 7.00 (dd, J=7.9 Hz, 1.7 Hz, H-12, 1H), 6.95 (dd, J=11.9 Hz, 1.7 Hz, H-14, 1H), 6.89-6.83 (m, H-5, 1H), 6.71-6.66 (m, H-4, 1H), 4.61 (d, J=14.3 Hz, H-6, 1H), 4.54 (d, J=14.3 Hz, H-6, 1H), 3.81 (d, J=14.1 Hz, H-7, 1H), 3.65-3.55 (m, H-7, H-10, 2H), 1.33 (d, J=7.1 Hz, H-11, 3H). ¹³C NMR (100 MHz, CD₃OD) δ 178.5, 165.9 (dd, ¹J_{C-F}=247.3 Hz, ³J_{C-F}=11.9 Hz), 161.0 (d, ¹J_{C-F}=246.2 Hz), 160.7 (dd, ¹J_{C-F}=247.3 Hz, ³J_{C-F}=11.9 Hz), 151.5, 146.4, 144.4, 137.0, 131.9, 131.5, 130.1, 129.7, 129.1, 128.9, 125.3, 124.7, 116.0, 112.0, 104.9, 77.2, 57.3, 47.6, 46.5, 18.5. ¹⁹F NMR (375 MHz, CD₃OD) δ+−109.87 (m, Fₚₐᵣₐ), −113.01 (m, Fₒᵣₜₕₒ), −119.87 (m, Fₘₑₜₐ).

Hybrids 9 and 12. S-Naproxen (118 mg, 0.51 mmol) was dissolved in dry DMF (2 mL) under argon at 0° C. then treated with HATU (330 mg, 0.87 mmol) and DIPEA (0.30 mL, 1.72 mmol) and stirred for 10 min at 0° C. To the reaction mixture, racemate 1b (106 mg, 0.42 mmol) was added, and the solution was stirred at room temperature. The reaction was monitored by TLC (MeOH/DCM, 1:9). Upon completion at 3 h, the product was extracted with ethyl acetate, washed with H₂O, dried over MgSO₄, and concentrated to give the crude diastereomers. The concentrated crude was purified by column chromatography on SiO₂ using a gradient of MeOH/DCM as eluent to afford the diastereomer mix. The diastereomers were separated by preparative RP-HPLC to afford hybrids 9 and 12.

Hybrid 9 (60 mg, 62%). HRESI-MS m/z calculated for C₂₅H₂₅F₂N₄O₃, 467.1895; found for [M+H]⁺, 467.1894. ¹H NMR (500 MHz, CD₃OD) δ 8.28 (s, H-2, 1H), 7.78 (s, H-1, 1H), 7.68 (d, J=9.0 Hz, H-14, 1H), 7.65 (d, J=8.6 Hz, H-15, 1H), 7.56 (s, H-12, 1H), 7.30-7.24 (m, H-3, 1H), 7.22-7.19 (m, H-13, H-17, 2H), 7.13 (dd, J=9.2 Hz, 2.5 Hz, H-16, 1H), 6.83-6.78 (m, H-5, 1H), 6.53-6.49 (m, H-4, 1H), 4.66 (d, J=14.3 Hz, H-6, 1H), 4.55 (d, J=14.4 Hz, H-6, 1H), 3.95-3.91 (m, H-7, H-18, 4H), 3.37 (q, J=7.1 Hz, H-10, 1H), 3.51 (d, J=14.1 Hz, H-7, 1H), 1.40 (d, J=7.1 Hz, H-11, 3H). ¹³C NMR (125 MHz, CD₃OD) δ 178.6, 163.2 (dd, ¹J_{C-F}=245.0 Hz, ³J_{C-F}=11.1 Hz), 160.5 (dd, ¹J_{C-F}=245.9 Hz, ³J_{C-F}=13.2 Hz), 159.2, 151.4, 146.2, 137.6, 135.3, 131.4, 130.4, 130.3, 128.2, 127.0, 126.8, 125.0, 120.0, 111.8, 106.7, 104.9, 76.7, 57.1, 55.8, 47.7, 47.2, 18.7. ¹⁹F NMR (470 MHz, CD₃OD) δ+−109.29 (m, Fₚₐᵣₐ), −112.99 (m, Fₒᵣₜₕₒ).

Hybrid 12 (42 mg, 43%). HRESI-MS m/z calculated for C₂₅H₂₅F₂N₄O₃, 467.1895; found for [M+H]⁺, 467.1892. ¹H NMR (500 MHz, CD₃OD) δ 8.27 (s, H-2, 1H), 7.78 (s, H-1, 1H), 7.68 (d, J=9.1 Hz, H-14, 1H), 7.64 (d, J=8.5 Hz, H-15, 1H), 7.54 (s, H-12, 1H), 7.22-7.12 (m, H-3, H-13, H-16, H-17, 4H), 6.80-6.74 (m, H-5, 1H), 6.44-6.39 (m, H-4, 1H), 4.57 (d, J=14.2 Hz, H-6, 1H), 4.49 (d, J=14.3 Hz, H-6, 1H), 3.93 (s, H-18, 3H), 3.75 (d, J=14.3 Hz, H-7, 1H), 3.70-3.65 (m, H-7, H-10, 2H), 1.41 (d, J=7.1 Hz, H-11, 3H). ¹³C NMR (125 MHz, CD₃OD) b 179.3, 164.2 (dd, J_{C-F}=246.8 Hz, ³J_{C-F}=11.4 Hz), 160.5 (dd, ¹J_{C-F}=246.8 Hz, ³J_{C-F}=12.6 Hz), 159.3, 151.4, 146.3, 137.7, 135.3, 131.4, 130.4, 130.3, 128.3, 127.0, 126.8, 125.1, 120.0, 111.9, 106.7, 104.8, 77.2, 57.1, 55.8, 48.1, 47.0, 18.3. ¹⁹F NMR (470 MHz, CD₃OD) δ+−109.48 (m, Fₚₐᵣₐ), −113.05 (m, Fₒᵣₜₕₒ).

Hybrids 10 and 11. R-Naproxen (138 mg, 0.60 mmol) was dissolved in dry DMF (2 mL) under argon at 0° C. then treated with HATU (375 mg, 0.99 mmol) and DIPEA (0.34 mL, 1.95 mmol) and stirred for 10 min at 0° C. To the reaction mixture, racemate 1b (122 mg, 0.48 mmol) was added, and the solution was stirred at room temperature. The reaction was monitored by TLC (MeOH/DCM, 1:9). Upon completion at 3 h, the product was extracted with ethyl acetate, washed with H₂O, dried over MgSO₄, and concentrated to give the crude diastereomers. The concentrated crude was purified by column chromatography on SiO₂ using a gradient of MeOH/DCM as eluent to afford the diastereomer mix. The diastereomers were separated by preparative RP-HPLC to afford hybrids 10 and 11.

Hybrid 10 (41 mg, 35%). HRESI-MS m/z calculated for C₂₅H₂₅F₂N₄O₃, 467.1895; found for [M+H]⁺, 467.1896. ¹H NMR (500 MHz, CD₃OD) δ 8.25 (s, H-2, 1H), 7.77 (s, H-1, 1H), 7.65 (d, J=9.0 Hz, H-14, 1H), 7.62 (d, J=8.5 Hz, H-15, 1H), 7.52 (s, H-12, 1H), 7.20-7.11 (m, H-3, H-13, H-16, H-17, 4H), 6.78-6.73 (m, H-5, 1H), 6.42-6.37 (m, H-4, 1H), 4.55 (d, J=14.5 Hz, H-6, 1H), 4.47 (d, J=14.5 Hz, H-6, 1H), 3.91 (s, H-18, 3H), 3.73 (d, J=14.5 Hz, H-7, 1H), 3.68-3.63 (m, H-7, H-10, 2H), 1.39 (d, J=7.1 Hz, H-11, 3H). ¹³C NMR (125 MHz, CD₃OD) δ 179.2, 164.2 (dd, ¹J_{C-F}=247.3 Hz, ³J_{C-F}=12.3 Hz), 160.4 (dd, ¹J_{C-F}=246.8 Hz, ³J_{C-F}=12.3 Hz), 159.2, 151.4, 146.2, 137.7, 135.3, 131.4, 130.4, 130.3, 128.2, 127.0, 126.8, 125.1, 120.0, 111.9, 106.7, 104.7, 77.1, 57.1, 55.8, 48.1, 47.0, 18.3. ¹⁹F NMR (470 MHz, CD₃OD) δ+−109.81 (m, Fₚₐᵣₐ), −113.01 (m, Fₒᵣₜₕₒ).

Hybrid 11 (59 mg, 50%). HRESI-MS m/z calculated for C₂₅H₂₅F₂N₄O₃, 467.1895; found for [M+H]⁺, 467.1893. ¹H NMR (500 MHz, CD₃OD) δ 8.27 (s, H-2, 1H), 7.77 (s, H-1, 1H), 7.67 (d, J=8.9 Hz, H-14, 1H), 7.63 (d, J=8.6 Hz, H-15, 1H), 7.54 (s, H-12, 1H), 7.28-7.23 (m, H-3, 1H), 7.20-7.18 (m, H-13, H-17, 2H), 7.12 (dd, J=8.9 Hz, 2.3 Hz, H-16, 1H), 6.82-6.77 (m, H-5, 1H), 6.52-6.47 (m, H-4, 1H), 4.64 (d, J=13.8 Hz, H-6, 1H), 4.54 (d, J=14.2 Hz, H-6, 1H), 3.90-3.93 (m, H-7, H-18, 4H), 3.66 (q, J=6.9 Hz, H-10, 1H), 3.50 (d, J=14.4 Hz, H-7, 1H), 1.39 (d, J=7.1 Hz, H-11, 3H). ¹³C NMR (125 MHz, CD₃OD) δ 178.6, 164.2 (dd, ¹J_{C-F}=247.6 Hz, ³J_{C-F}=11.8 Hz), 160.7 (dd, ¹J_{C-F}=247.6 Hz, ³J_{C-F}=11.8 Hz), 159.2, 151.4, 146.2, 137.6, 135.3, 131.4, 130.4, 130.3, 128.2, 127.0, 126.8, 125.0, 120.0, 111.8, 106.7, 104.9, 76.7, 57.1, 55.8, 47.7, 47.2, 18.7. ¹⁹F NMR (470 MHz, CD₃OD) δ+−109.27 (m, Fₚₐᵣₐ), −112.96 (m, Fₒᵣₜₕₒ).

Hybrids 13 and 14. Ketoprofen (122 mg, 0.48 mmol) was dissolved in dry DMF (2 mL) under argon at 0° C. then treated with HATU (311 mg, 0.82 mmol) and DIPEA (0.28 mL, 1.61 mmol) and stirred for 10 min at 0° C. To the reaction mixture, 1b-(S) (102 mg, 0.40 mmol) was added, and the solution was stirred at room temperature. The reaction was monitored by TLC (MeOH/DCM, 1:9). Upon completion at 3 h, the product was extracted with ethyl acetate, washed with H₂O, dried over MgSO₄, and concentrated to give the crude diastereomers. The concentrated crude was purified by column chromatography on SiO₂ using a gradient of MeOH/DCM as eluent to afford the diastereomer mix. The diastereomers were separated by preparative RP-HPLC to afford hybrids 13 and 14.

Hybrid 13 (62 mg, 63%). HRESI-MS m/z calculated for C₂₇H₂₄F₂N₄O₃Na, 513.1714; found for [M+Na]⁺, 513.1713. ¹H NMR (500 MHz, CD₃OD) δ 8.31 (s, H-2, 1H), 7.78-7.75 (m, H-1, H-16, 3H), 7.67-7.60 (m, H-12, H-15, H-18, 3H), 7.55-7.52 (m, H-17, 2H), 7.44-7.39 (m, H-13, H-14, 2H), 7.36-7.31 (m, H-3, 1H), 6.85-6.80 (m, H-5, 1H), 6.69-6.65 (m, H-4, 1H), 4.67 (d, J=14.3 Hz, H-6, 1H), 4.57 (d, J=14.2 Hz, H-6, 1H), 3.94 (d, J=14.2 Hz, H-7, 1H), 3.64 (q, J=7.1 Hz H-10, 1H), 3.49 (d, J=14.1 Hz, H-7, 1H), 1.34 (d, J=7.1 Hz, H-11, 3H). ¹³C NMR (125 MHz, CD₃OD) δ 198.5, 177.9, 164.3 (dd, ¹J_{C-F}=248.6 Hz, ³J_{C-F}=12.4 Hz), 160.9 (dd, ¹J_{C-F}=246.6 Hz, ³J_{C-F}=11.9 Hz), 151.6, 146.3, 143.3, 139.1, 139.0, 134.0, 132.9, 131.5, 131.2, 130.2, 130.0, 129.8, 129.7, 125.2, 112.0, 105.0, 76.8, 57.2, 47.8, 47.0, 19.0. ¹⁹F NMR (470 MHz, CD₃OD) δ+−109.08 (m, Fₚₐᵣₐ), −112.92 (m, Fₒᵣₜₕₒ).

Hybrid 14 (61 mg, 62%). HRESI-MS m/z calculated for C₂₇H₂₅F₂N₄O₃, 491.1895; found for [M+H]⁺, 491.1890. ¹H NMR (500 MHz, CD₃OD) δ 8.32 (s, H-2, 1H), 7.78-7.74 (m, H-1, H-16, 3H), 7.67-7.60 (m, H-12, H-15, H-18, 3H), 7.55-7.52 (m, H-17, 2H), 7.44-7.38 (m, H-13, H-14, 2H), 7.29-7.24 (m, H-3, 1H), 6.85-6.80 (m, H-5, 1H), 6.66-6.61 (m, H-4, 1H), 4.60 (d, J=14.2 Hz, H-6, 1H), 4.51 (d, J=14.2 Hz, H-6, 1H), 3.76 (d, J=14.2 Hz, H-7, 1H), 3.67-3.61 (m, H-7, H-10, 2H), 1.34 (d, J=7.1 Hz, H-11, 3H). ¹³C NMR (125 MHz, CD₃OD) δ 198.5, 178.6, 164.3 (dd, J_{C-F}=247.2 Hz, ³J_{C-F}=12.6 Hz), 160.7 (dd, ¹J_{C-F}=246.4 Hz, ³J_{C-F}=11.7 Hz), 151.5, 146.4, 143.4, 139.1, 139.0, 134.0, 132.8, 131.5, 131.2, 130.1, 130.0, 129.9, 129.7, 125.3, 112.1, 105.0, 77.2, 57.2, 48.2, 46.9, 18.6. ¹⁹F NMR (470 MHz, CD₃OD) δ+−109.57 (m, Fₚₐᵣₐ), −112.92 (m, Fₒᵣₜₕₒ).

Hybrids 15 and 16. Ketoprofen (122 mg, 0.48 mmol) was dissolved in dry DMF (2 mL) under argon at 0° C. then treated with HATU (303 mg, 0.80 mmol) and DIPEA (0.30 mL, 1.72 mmol) and stirred for 10 min at 0° C. To the reaction mixture, 1b-(R) (102 mg, 0.40 mmol) was added, and the solution was stirred at room temperature. The reaction was monitored by TLC (MeOH/DCM, 1:9). Upon completion at 3 h, the product was extracted with ethyl acetate, washed with H₂O, dried over MgSO₄, and concentrated to give the crude diastereomers. The concentrated crude was purified by column chromatography on SiO₂ using a gradient of MeOH/DCM as eluent to afford the diastereomer mix. The diastereomers were separated by preparative RP-HPLC to afford hybrids 15 and 16.

Hybrid 15 (84 mg, 86%). HRESI-MS m/z calculated for C₂₇H₂₅F₂N₄O₃, 491.1895; found for [M+H]⁺, 491.1897. ¹H NMR (500 MHz, CD₃OD) δ 8.31 (s, H-2, 1H), 7.79-7.76 (m, H-1, H-16, 3H), 7.68-7.61 (m, H-12, H-15, H-18, 3H), 7.56-7.53 (m, H-17, 2H), 7.44-7.39 (m, H-13, H-14, 2H), 7.36-7.31 (m, H-3, 1H), 6.86-6.81 (m, H-5, 1H), 6.70-6.66 (m, H-4, 1H), 4.68 (d, J=14.3 Hz, H-6, 1H), 4.58 (d, J=14.3 Hz, H-6, 1H), 3.94 (d, J=14.2 Hz, H-7, 1H), 3.64 (q, J=7.0 Hz, H-10, 1H), 3.50 (d, J=14.1 Hz, H-7, 1H), 1.34 (d, J=7.0 Hz, H-11, 3H). ¹³C NMR (125 MHz, CD₃OD) δ 198.4, 177.9, 164.2 (dd, ¹J_{C-F}=247.2 Hz, ³J_{C-F}=12.5 Hz), 160.8 (dd, ¹J_{C-F}=247.3 Hz, ³J_{C-F}=11.5 Hz), 151.5, 146.2, 143.2, 139.0, 138.9, 134.0, 132.9, 131.4, 131.1, 130.1, 129.9, 129.6, 129.6, 125.1, 112.0, 105.0, 76.7, 57.1, 47.7, 46.9, 18.9. ¹⁹F NMR (470 MHz, CD₃OD) δ+−109.10 (m, Fₚₐᵣₐ), −112.94 (m, Fₒᵣₜₕₒ).

Hybrid 16 (72 mg, 73%). HRESI-MS m/z calculated for C₂₇H₂₄F₂N₄O₃Na, 513.1714; found for [M+Na]⁺, 513.1717. ¹H NMR (400 MHz, CD₃OD) δ 8.32 (s, H-2, 1H), 7.79-7.74 (m, H-1, H-16, 3H), 7.68-7.60 (m, H-12, H-15, H-18, 3H), 7.56-7.51 (m, H-17, 2H), 7.45-7.38 (m, H-13, H-14, 2H), 7.30-7.24 (m, H-3, 1H), 6.86-6.80 (m, H-5, 1H), 6.66-6.61 (m, H-4, 1H), 4.60 (d, J=14.3 Hz, H-6, 1H), 4.51 (d, J=14.3 Hz, H-6, 1H), 3.76 (d, J=14.3 Hz, H-7, 1H), 3.67-3.61 (m, H-7, H-10, 2H), 1.34 (d, J=7.1 Hz, H-11, 3H). ¹³C NMR (100 MHz, CD₃OD) δ 198.4, 178.5, 164.2 (dd, ¹J_{C-F}=247.2 Hz, ³J_{C-F}=12.3 Hz), 160.6 (dd, ¹J_{C-F}=247.2 Hz, ³J_{C-F}=12.3 Hz), 151.4, 146.3, 143.3, 139.0, 138.9, 133.9, 132.7, 131.4, 131.1, 130.0, 129.9, 129.8, 129.6, 125.2, 111.9, 104.9, 77.1, 57.1, 48.1, 46.8, 18.5. ¹⁹F NMR (375 MHz, CD₃OD) δ+−109.58 (m, Fₚₐᵣₐ), −112.95 (m, Fₒᵣₜₕₒ).

Hybrid 17. Niflumic acid (72 mg, 0.26 mmol) was dissolved in dry DMF (2 mL) under argon at 0° C. then treated with HATU (152 mg, 0.40 mmol) and DIPEA (0.14 mL, 0.80 mmol) and stirred for 10 min at 0° C. To the reaction mixture, 1b-(S) (50 mg, 0.20 mmol) was added, and the solution was stirred at room temperature. The reaction was monitored by TLC (MeOH/DCM, 1:9). Upon completion at 3 h, the product was extracted with ethyl acetate, washed with H₂O, dried over MgSO₄, and concentrated to give the crude enantiomer. The concentrated crude was purified first by flash column chromatography on SiO₂ using a gradient of MeOH/DCM as eluent and then by preparative RP-HPLC to afford hybrid 17 (83 mg, 81%). HRESI-MS m/z calculated for C₂₄H₁₉F₅N₆O₂Na, 541.1387; found for [M+Na]⁺, 541.1383. ¹H NMR (400 MHz, CD₃OD) δ 8.36 (s, H-2, 1H), 8.27 (dd, J=4.9, 1.8 Hz, H-12, 1H), 8.16 (s, H-14, 1H), 7.84 (dd, J=7.8, 1.8 Hz, H-10, 1H), 7.78 (s, H-1, 1H), 7.68 (d, J=8.2 Hz, H-17, 1H), 7.53-7.46 (m, H-3, 1H), 7.42 (t, J=8.0 Hz, H-16, 1H), 7.21 (d, J=7.7 Hz, H-15, 1H), 6.97-6.91 (m, H-5, 1H), 6.82-6.77 (m, H-4, H-11, 2H), 4.82 (d, J=14.4 Hz, H-6, 1H), 4.69 (d, J=14.4 Hz, H-6, 1H), 3.98 (d, J=14.2 Hz, H-7, 1H), 3.87 (d, J=14.1 Hz, H-7, 1H). ¹³C NMR (100 MHz, CD₃OD) δ 169.8, 163.0 (dd, ¹J_{C-F}=247.7 Hz, ³J_{C-F}=12.3 Hz), 159.6 (dd, ¹J_{C-F}=246.8 Hz, ³J_{C-F}=12.3 Hz), 154.3, 150.6, 150.1, 144.9, 141.0, 136.7, 130.7 (q, ³J_{CF3}=31.7 Hz), 130.0, 129.1, 124.4 (d, ¹J_{CF3}=272.0 Hz), 124.1, 122.6, 117.8, 115.6, 113.9, 111.8, 110.6, 103.7, 75.6, 55.6, 46.6. ¹⁹F NMR (375 MHz, CD₃OD) δ+−64.16 (s, CF₃), −108.55 (m, Fₚₐᵣₐ), −112.86 (m, Fₒᵣₜₕₒ).

Hybrid 18. Niflumic acid (67 mg, 0.24 mmol) was dissolved in dry DMF (2 mL) under argon at 0° C. then treated with HATU (152 mg, 0.40 mmol) and DIPEA (0.14 mL, 0.80 mmol) and stirred for 10 min at 0° C. To the reaction mixture, 1b-(R) (50 mg, 0.20 mmol) was added, and the solution was stirred at room temperature. The reaction was monitored by TLC (MeOH/DCM, 1:9). Upon completion at 3 h, the product was extracted with ethyl acetate, washed with H₂O, dried over MgSO₄, and concentrated to give the crude enantiomer. The concentrated crude was first purified by flash column chromatography on SiO₂ using a gradient of MeOH/DCM as eluent and then by preparative RP-HPLC to afford hybrid 18 (90 mg, 89%). HRESI-MS m/z calculated for C₂₄H₂₀F₅N₆O₂, 519.1568; found for [M+H]⁺, 519.1564. ¹H NMR (500 MHz, CD₃OD) δ 8.39 (s, H-2, 1H), 8.30 (dd, J=4.8, 1.7 Hz, H-12, 1H), 8.19 (s, H-14, 1H), 7.87 (dd, J=7.7, 1.6 Hz, H-10, 1H), 7.81 (s, H-1, 1H), 7.72 (d, J=8.1 Hz, H-17, 1H), 7.55-7.50 (m, H-3, 1H), 7.46 (t, J=8.0 Hz, H-16, 1H), 7.25 (d, J=7.7 Hz, H-15, 1H), 7.00-6.95 (m, H-5, 1H), 6.85-6.81 (m, H-4, H-11, 2H), 4.85 (d, J=14.3 Hz, H-6, 1H), 4.73 (d, J=14.3 Hz, H-6, 1H), 4.02 (d, J=14.1 Hz, H-7, 1H), 3.90 (d, J=14.1 Hz, H-7, 1H). ¹³C NMR (125 MHz, CD₃OD) δ 171.7, 164.8 (dd, ¹J_{C-F}=248.4 Hz, ³J_{C-F}=12.7 Hz), 161.5 (dd, ¹J_{C-F}=246.8 Hz, ³J_{C-F}=12.1 Hz), 156.2, 152.4, 151.9, 146.7, 142.8, 138.6, 132.5 (q, ³J_{CF3}=31.9 Hz), 131.9, 130.9, 126.3 (d, ¹J_{CF3}=271.6 Hz), 125.9, 124.4, 119.6, 117.5, 115.7, 113.6, 112.5, 105.5, 77.4, 57.5, 48.4. ¹⁹F NMR (470 MHz, CD₃OD) δ+−64.19 (s, CF₃), −108.57 (m, Fₚₐᵣₐ), −112.88 (m, Fₒᵣₜₕₒ).

Hybrid 19. Diflunisal (63 mg, 0.25 mmol) was dissolved in dry DMF (2 mL) under argon at 0° C. then treated with HATU (151 mg, 0.40 mmol) and stirred for 10 min at 0° C. To the reaction mixture, 1b-(S) (50 mg, 0.20 mmol) was added, and the solution was stirred at room temperature. The reaction was monitored by TLC (MeOH/DCM, 1:9). Upon completion at 3 h, the product was extracted with ethyl acetate, washed with H₂O, dried over MgSO₄, and concentrated to give the crude enantiomer. The concentrated crude was first purified by flash column chromatography on SiO₂ using a gradient of MeOH/DCM as eluent and then by preparative RP-HPLC to afford hybrid 19 (22 mg, 23%). HRESI-MS m/z calculated for C₂₄H₁₈F₄N₄O₃Na, 509.1213; found for [M+Na]⁺, 509.1207. ¹H NMR (500 MHz, CD₃OD) δ 8.35 (s, H-2, 1H), 7.90 (dd, J=2.2, 1.0 Hz, H-13, 1H), 7.77 (s, H-1, 1H), 7.53-7.48 (m, H-12, H-14, 2H), 7.41-7.46 (m, H-3, 1H), 7.03-6.98 (m, H-15, H-16, 2H), 6.97-6.92 (m, H-5, H-11, 2H), 6.86-6.82 (m, H-4, 1H), 4.81 (d, J=14.4 Hz, H-6, 1H), 4.68 (d, J=14.4 Hz, H-6, 1H), 4.02 (d, J=14.1 Hz, H-7, 1H), 3.94 (d, J=14.4 Hz, H-7, 1H). ¹³C NMR (125 MHz, CD₃OD) δ 171.0, 164.6 (dd, ¹J_{C-F}=248.0 Hz, ³J_{C-F}=12.4 Hz), 163.8 (dd, ¹J_{C-F}=247.5 Hz, ³J_{C-F}=12.0 Hz), 161.3 (dd, ¹J_{C-F}=248.8 Hz, ³J_{C-F}=12.0 Hz), 161.1 (dd, ¹J_{C-F}=247.1 Hz, ³J_{C-F}=12.0 Hz), 160.0, 151.6, 146.4, 135.4, 132.7, 131.6, 130.8, 127.6, 126.1, 125.6, 118.7, 118.0, 112.8, 112.3, 105.2, 76.9, 57.3, 48.0. ¹⁹F NMR (470 MHz, CD₃OD) δ+−109.05 (m, Fₚₐᵣₐ), −112.97 (m, Fₒᵣₜₕₒ), −113.84 (m, Fₚₐᵣₐ), −115.49 (m, Fₒᵣₜₕₒ).

Hybrid 20. Diflunisal (60 mg, 0.24 mmol) was dissolved in dry DMF (2 mL) under argon at 0° C. then treated with HATU (152 mg, 0.40 mmol) and stirred for 10 min at 0° C. To the reaction mixture, 1b-(R) (50 mg, 0.20 mmol) was added, and the solution was stirred at room temperature. The reaction was monitored by TLC (MeOH/DCM, 1:9). Upon completion at 3 h, the product was extracted with ethyl acetate, washed with H₂O, dried over MgSO₄, and concentrated to give the crude enantiomer. The concentrated crude was first purified by flash column chromatography on SiO₂ using a gradient of MeOH/DCM as eluent and then by preparative RP-HPLC to afford hybrid 20 (31 mg, 32%). HRESI-MS m/z calculated for C₂₄H₁₈F₄N₄O₃Na, 509.1213; found for [M+Na]⁺, 509.1204. ¹H NMR (400 MHz, CD₃OD) δ 8.36 (s, H-2, 1H), 7.92-7.91 (m, H-13, 1H), 7.79 (s, H-1, 1H), 7.55-7.42 (m, H-3, H-12, H-14, 3H), 7.04-6.92 (m, H-5, H-11, H-15, H-16, 4H), 6.88-6.82 (m, H-4, 1H), 4.82 (d, J=14.3 Hz, H-6, 1H), 4.69 (d, J=14.3 Hz, H-6, 1H), 4.02 (d, J=14.0 Hz, H-7, 1H), 3.96 (d, J=14.2 Hz, H-7, 1H). ¹³C NMR (100 MHz, CD₃OD) δ 170.7, 164.5 (dd, ¹J_{C-F}=247.6 Hz, ³J_{C-F}=12.5 Hz), 163.7 (dd, ¹J_{C-F}=247.6 Hz, ³J_{C-F}=11.7 Hz), 161.0 (d, ¹J_{C-F}=247.0 Hz), 160.9 (d, ¹J_{C-F}=247.0 Hz), 159.6, 151.5, 146.2, 135.4, 132.7, 132.1, 130.7, 127.6, 125.8, 125.5, 118.4, 117.9, 112.7, 112.2, 105.2, 76.7, 57.2, 48.8. ¹⁹F NMR (375 MHz, CD₃OD) δ+−109.22 (m, Fₚₐᵣₐ), −113.12 (m, Fₒᵣₜₕₒ), −113.95 (m, Fₚₐᵣₐ), −115.66 (m, Fₒᵣₜₕₒ).

Hybrid 21. Salicylic acid (35 mg, 0.25 mmol) was dissolved in dry DMF (2 mL) under argon at 0° C. then treated with HATU (152 mg, 0.40 mmol) and stirred for 10 min at 0° C. To the reaction mixture, 1b-(S) (50 mg, 0.20 mmol) was added, and the solution was stirred at room temperature. The reaction was monitored by TLC (MeOH/DCM, 1:9). Upon completion at 3 h, the product was extracted with ethyl acetate, washed with H₂O, dried over MgSO₄, and concentrated to give the crude enantiomer. The concentrated crude was first purified by flash column chromatography on SiO₂ using a gradient of MeOH/DCM as eluent and then by preparative RP-HPLC to afford hybrid 21 (24 mg, 32%). HRESI-MS m/z calculated for C₁₈H₁₆F₂N₄O₃Na, 397.1088; found for [M+Na]⁺, 397.1081. ¹H NMR (400 MHz, CD₃OD) b 8.36 (s, H-2, 1H), 7.78 (s, H-1, 1H), 7.73 (dd, J=8.3, 1.7 Hz, H-10, 1H), 7.54-7.47 (m, H-3, 1H), 7.37-7.32 (m, H-12, 1H), 6.98-6.92 (m, H-5, 1H), 6.89-6.81 (m, H-4, H-11, H-13, 3H), 4.82 (d, J=14.4 Hz, H-6, 1H), 4.68 (d, J=14.4 Hz, H-6, 1H), 4.00 (d, J=14.0 Hz, H-7, 1H), 3.94 (d, J=14.2 Hz, H-7, 1H). ¹³C NMR (100 MHz, CD₃OD) δ 169.9, 163.1 (dd, ¹J_{C-F}=248.4 Hz, ³J_{C-F}=12.6 Hz), 159.6 (dd, ¹J_{C-F}=246.8 Hz, ³J_{C-F}=11.8 Hz), 158.8, 150.1, 144.9, 133.6, 130.1, 128.7, 124.2, 119.1, 116.9, 116.2, 110.8, 103.7, 75.4, 55.8, 46.4. ¹⁹F NMR (375 MHz, CD₃OD) δ −109.25 (m, Fₚₐᵣₐ), −113.21 (m, Fₒᵣₜₕₒ).

Hybrid 22. Salicylic acid (33 mg, 0.24 mmol) was dissolved in dry DMF (2 mL) under argon at 0° C. then treated with HATU (150 mg, 0.40 mmol) and stirred for 10 min at 0° C. To the reaction mixture, 1b-(R) (50 mg, 0.20 mmol) was added, and the solution was stirred at room temperature. The reaction was monitored by TLC (MeOH/DCM, 1:9). Upon completion at 3 h, the product was extracted with ethyl acetate, washed with H₂O, dried over MgSO₄, and concentrated to give the crude enantiomer. The concentrated crude was first purified by flash column chromatography on SiO₂ using a gradient of MeOH/DCM as eluent and then by preparative RP-HPLC to afford hybrid 22 (24 mg, 32%). HRESI-MS m/z calculated for C₁₈H₁₆F₂N₄O₃Na, 397.1088; found for [M+Na]⁺, 397.1089. ¹H NMR (400 MHz, CD₃OD) b 8.36 (s, H-2, 1H), 7.78 (s, H-1, 1H), 7.73 (dd, J=8.2, 1.7 Hz, H-10, 1H), 7.54-7.47 (m, H-3, 1H), 7.37-7.32 (m, H-12, 1H), 6.98-6.92 (m, H-5, 1H), 6.89-6.81 (m, H-4, H-11, H-13, 3H), 4.82 (d, J=14.3 Hz, H-6, 1H), 4.68 (d, J=14.4 Hz, H-6, 1H), 4.00 (d, J=14.2 Hz, H-7, 1H), 3.94 (d, J=14.1 Hz, H-7, 1H). 13C NMR (100 MHz, CD₃OD) δ 171.2, 164.4 (dd, ¹J_{C-F}=248.2 Hz, ³J_{C-F}=12.8 Hz), 160.9 (dd, ¹J_{C-F}=247.1 Hz, ³J_{C-F}=12.1 Hz), 160.1, 151.4, 146.2, 135.0, 131.5, 130.1, 125.5, 120.4, 118.2, 117.5, 112.1, 105.1, 76.7, 57.2, 47.8. ¹⁹F NMR (375 MHz, CD₃OD) δ −109.23 (m, Fₚₐᵣₐ), −113.19 (m, Fₒᵣₜₕₒ).

Hybrid 23. Diclofenac (72 mg, 0.24 mmol) was dissolved in dry DMF (2 mL) under argon at 0° C. then treated with HATU (152 mg, 0.40 mmol) and stirred for 10 min at 0° C. To the reaction mixture, 1b-(S) (51 mg, 0.20 mmol) was added, and the solution was stirred at room temperature. The reaction was monitored by TLC (MeOH/DCM, 1:9). Upon completion at 3 h, the product was extracted with ethyl acetate, washed with H₂O, dried over MgSO₄, and concentrated to give the crude enantiomer. The concentrated crude was first purified by flash column chromatography on SiO₂ using a gradient of MeOH/DCM as eluent and then by preparative RP-HPLC to afford hybrid 23 (50 mg, 48%). HRESI-MS m/z calculated for C₂₅H₂₁Cl₂F₂N₅O₂Na, 554.0938; found for [M+Na]⁺, 554.0944. ¹H NMR (400 MHz, CD₃OD) δ 8.33 (s, H-2, 1H), 7.79 (s, H-1, 1H), 7.41 (d, J=8.2 Hz, H-16, 2H), 7.37-7.30 (m, H-3, 1H), 7.10-6.98 (m, H-11, H-13, H-17, 3H), 6.87-6.78 (m, H-5, H-12, 2H), 6.61-6.55 (m, H-4, 1H), 6.33 (d, J=7.9 Hz, H-14, 1H), 4.66 (d, J=14.3 Hz, H-6, 1H), 4.59 (d, J=14.4 Hz, H-6, 1H), 3.81 (d, J=14.3 Hz, H-7, 1H), 3.67 (d, J=14.4 Hz, H-7, 1H), 3.58 (d, J=13.8 Hz, H-10, 1H), 3.53 (d, J=13.6 Hz, H-10, 1H). ¹³C NMR (100 MHz, CD₃OD) δ 176.5, 164.2 (dd, ¹J_{C-F}=248.3 Hz, ³J_{C-F}=12.3 Hz), 160.6 (dd, ¹J_{C-F}==246.4 Hz, ³J_{C-F}==12.3 Hz), 151.4, 146.3, 144.4, 139.2, 131.5, 131.3, 130.1, 128.7, 126.4, 125.7, 125.0, 122.5, 118.1, 112.0, 104.9, 76.9, 57.1, 48.2, 40.4. ¹⁹F NMR (375 MHz, CD₃OD) δ+−109.69 (m, Fₚₐᵣₐ), −113.00 (m, Fₒᵣₜₕₒ).

Hybrid 24. Diclofenac (67 mg, 0.23 mmol) was dissolved in dry DMF (2 mL) under argon at 0° C. then treated with HATU (144 mg, 0.38 mmol) and stirred for 10 min at 0° C. To the reaction mixture, 1b-(R) (50 mg, 0.20 mmol) was added, and the solution was stirred at room temperature. The reaction was monitored by TLC (MeOH/DCM, 1:9). Upon completion at 3 h, the product was extracted with ethyl acetate, washed with H₂O, dried over MgSO₄, and concentrated to give the crude enantiomer. The concentrated crude was first purified by flash column chromatography on SiO₂ using a gradient of MeOH/DCM as eluent and then by preparative RP-HPLC to afford hybrid 24 (52 mg, 50%). HRESI-MS m/z calculated for C₂₅H₂₁Cl₂F₂N₅O₂Na, 554.0938; found for [M+Na]⁺, 554.0940. ¹H NMR (400 MHz, CD₃OD) δ 8.33 (s, H-2, 1H), 7.79 (s, H-1, 1H), 7.41 (d, J=8.1 Hz, H-16, 2H), 7.37-7.30 (m, H-3, 1H), 7.10-6.98 (m, H-11, H-13, H-17, 3H), 6.87-6.78 (m, H-5, H-12, 2H), 6.61-6.55 (m, H-4, 1H), 6.33 (d, J=7.9 Hz, H-14, 1H), 4.67 (d, J=14.3 Hz, H-6, 1H), 4.59 (d, J=14.3 Hz, H-6, 1H), 3.81 (d, J=14.3 Hz, H-7, 1H), 3.67 (d, J=14.3 Hz, H-7, 1H), 3.58 (d, J=13.7 Hz, H-10, 1H), 3.53 (d, J=13.7 Hz, H-10, 1H). ¹³C NMR (100 MHz, CD₃OD) δ 176.5, 164.2 (dd, ¹J_{C-F}=247.2 Hz, ³J_{C-F}=12.0 Hz), 160.6 (dd, ¹J_{C-F}=246.0 Hz, ³J_{C-F}=12.03 Hz), 151.4, 146.3, 144.4, 139.2, 131.5, 131.3, 130.1, 128.7, 126.4, 125.7, 125.0, 122.5, 118.1, 112.0, 104.9, 76.9, 57.1, 48.2, 40.4. ¹⁹F NMR (375 MHz, CD₃OD) δ+−109.68 (m, Fₚₐᵣₐ), −112.94 (m, Fₒᵣₜₕₒ).

### Biological Assays

Preparation of stock solutions of the tested compounds: hybrids 1-24 were dissolved in anhydrous DMSO to final concentrations of 5 mg/mL. The antifungal drugs FLC and VOR were purchased from Sigma Aldrich were dissolved in anhydrous DMSO to final concentrations of 5 mg/mL.

Minimal inhibitory concentration broth double-dilution assay: C. auris minimal inhibitory concentrations (MICs) were determined using CLSI M27-A3 guidelines with minor modifications. Starter cultures were streaked from glycerol stock onto YPAD agar plates and grown for 24 h at 37° C. Colonies were suspended in 1 mL PBS and diluted to 1×10⁻³ optical density at 600 nm (OD₆₀₀) and then diluted 1:100 into fresh medium. hybrids dissolved in DMSO were added to YPAD broth (32 μL stock solution in 1218 μL of YPAD broth), and serial double dilutions of hybrids in YPAD were prepared in flat-bottomed 96-well microplates (Corning) to enable testing of concentrations ranging from 64 μg/mL to 0.007 μg/mL. Control wells with yeast cells but no drug and blank wells containing only YPAD were prepared. An equal volume (100 μL) of yeast suspension in YPAD broth was added to each well with the exceptions of the blank wells. After incubation for 24 h at 37° C., MTT (50 μL of a 1 mg/mL solution in ddH₂O) was added to each well followed by additional incubation at 37° C. for 2 h. MIC values (Table S3) were defined as the lowest concentration of an antifungal agent that caused a specified reduction in visible growth as per the CLSI M27-A3 protocol. The magnitude of reduction in visible growth was assessed using the following numerical scale: 0, optically clear; 1, slightly hazy; 2, prominent decrease (˜50%) in visible growth; 3, slight reduction in visible growth; and 4, no reduction in visible growth. The MIC was defined based on reduction in growth to 0 or 1. Results were confirmed in two independent experiments, and each concentration was tested in triplicate. FLC and VOR were used as control drugs.

TABLE S3

| | C. auris | C. auris |
|---|---|---|
| Compound | 5001 | 5002 |
| FLC | 8 | >64 |
| VOR | 0.03 | 1 |
| 1 | 0.06 | 1 |
| 2 | 0.06 | 1 |
| 5 | 0.06 | 4 |
| 10 | 0.5 | 16 |
| 17 | 0.25 | 4 |
| 19 | 0.12 | 2 |
| 21 | 0.25 | 8 |
| 23 | 2 | 64 |

Table S3 shows MIC values of selected hybrids against C. auris. Cells were grown in YPAD medium at 37° C. for 24 h.

C. albicans, C. glabrata, C. parapsilosis, C. guilliermondii, C. tropicalis, and C. dubliniensis MICs were determined using CLSI M27-A3 guidelines with minor modifications. Starter cultures were streaked from glycerol stock onto YPAD agar plates and grown for 24 h at 30° C. Colonies were suspended in 1 mL PBS and diluted to 1×10⁻³ OD₆₀₀ and then diluted 1:100 into fresh medium. Hybrids dissolved in DMSO were added to YPAD broth (32 μL stock solution in 1218 μL of YPAD broth), and serial double dilutions of hybrids in YPAD were prepared in flat-bottomed 96-well microplates (Corning) to enable testing of concentrations ranging from 64 μg/mL to 0.003 μg/mL. Control wells with yeast cells but no drug and blank wells containing only YPAD were prepared. An equal volume (100 μL) of yeast suspensions in YPAD broth was added to each well with the exceptions of the blank wells. MIC values (Tables S4-S6) were determined after 24 h at 30° C. by measuring the OD₆₀₀ using a plate reader (Infinite M200 PRO, Tecan). MIC values were defined as the point at which the OD₆₀₀ was reduced by >80% compared to the no-drug wells. Each concentration was tested in triplicate, and results were confirmed by two independent sets of experiments. FLC and VOR were used as control drugs.

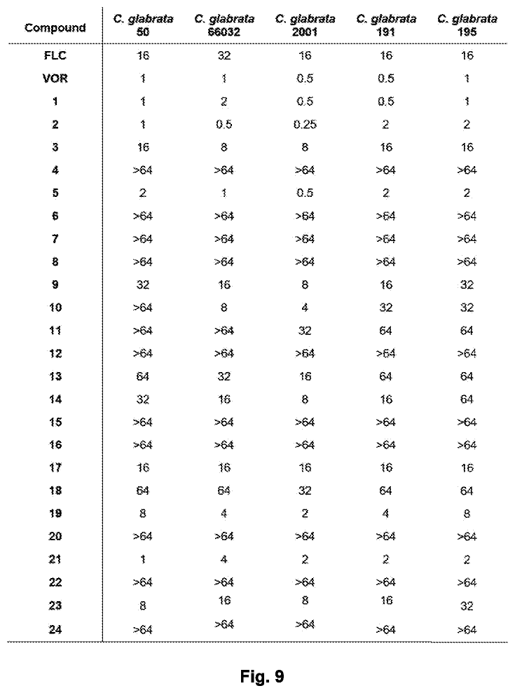

FIG. 9 shows table S5 which provides the MIC values of hybrids 1-24 against C. glabrata. Cells were grown in YPAD medium at 30° C. for 24 h. Values are given in μg/mL.

Disk diffusion assay: Antifungal activities of select hybrids against C. albicans SN152, C. parapsilosis ATCC 22019, and C. tropicalis 660 were confirmed by the disk diffusion assay. Strains were streaked from frozen culture onto YPAD agar and incubated for 24 h at 30° C. Two or three colonies were placed into 1 mL of PBS solution, and OD₆₀₀ was determined with a TECAN Infinite. OD₆₀₀ was adjusted to 0.02 for C. albicans SN152 and to 0.025 for C. parapsilosis ATCC 22019 and C. tropicalis 660 by dilution with PBS. Aliquots of 200 μL of the diluted cultures of each strain were plated onto 15-mL casitone agar plates and spread using sterile beads (3 mm, Fisher Scientific). After the plates dried, a single disk (6-mm diameter, Becton Dickinson) with 25 μg of the hybrid being tested was placed in the center of each plate. Plates were then incubated at 30° C. and photographed under the same imaging conditions after 24 h and 48 h. FLC and VOR were used as control drugs.

Growth curve analyses: Growth curves were determined using the double-dilution method in 96-well plates. Starter cultures were streaked from glycerol stock onto YPAD agar plates and grown for 24 h at 30° C. Colonies were suspended in 1 mL PBS and diluted to 1×10⁻³ OD₆₀₀ and then diluted 1:100 into fresh medium. Hybrids dissolved in DMSO were added to YPAD broth (32 μL stock solution in 1218 μL of YPAD broth), and serial double dilutions of hybrids in YPAD were prepared in flat-bottomed 96-well microplates (Corning) to enable testing of concentrations ranging from 64 μg/mL to 1 μg/mL. Control wells with yeast cells but no drug (100% growth) and blank wells containing only YPAD (0% growth) were prepared. An equal volume (100 μL) of yeast suspensions in YPAD broth was added to each well with the exceptions of the blank wells. Growth was determined at 30° C. by measuring the OD₆₀₀ using a plate reader (Infinite M200 PRO, Tecan) every 40 minutes over 48 h. Each concentration was tested in triplicate, and results were confirmed by two independent sets of experiments. FLC and VOR were used as control drugs.

In another embodiment, the present disclosure provides a composition for controlling or preventing plant fungal infections, growths, or diseases and/or horticultural crops and/or animals and/or insects comprising an effective amount of the antifungal compound and at least one additional component selected from the group consisting of surfactants and auxiliaries. In yet another embodiment, the composition additionally comprises at least one additional biologically active and compatible compound selected from fungicides, insecticides, nematicides, acaricides, biopesticides, herbicides, plant growth regulators, antibiotics, fertilizers or nutrients.

A pharmacological composition or formulation refers to a composition or formulation in a form suitable for administration, e.g., systemic administration, into subject, including for example a human. Suitable forms, in part, depend upon the use or the route of entry, for example oral, inhaled, transdermal, or by injection/infusion. Acceptable carriers, diluents, and/or excipients for therapeutic use are well known in the pharmaceutical art. In some embodiments, buffers, preservatives, bulking agents, dispersants, stabilizers, dyes, can be provided. In addition, antioxidants and suspending agents can be used.

1. An antifungal compound, an (R) enantiomer thereof, an (S) enantiomer thereof, a diastereomer thereof, a racemate thereof, or a salt thereof, comprising:
an azole pharmacophore coupled to an Ru moiety via a link;
wherein Ru is a COX inhibitor coupled to the azole pharmacophore via an amide bond between a primary amine of the azole pharmacophore and a carboxylic acid of the COX inhibitor; and
wherein the antifungal compound is according to Formula (I),

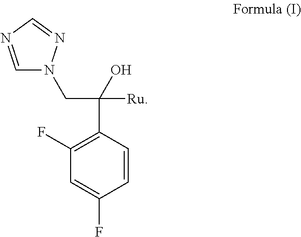

2. (canceled)

3. The antifungal compound as claimed in claim 1, wherein the azole pharmacophore is S-configured.

4. The antifungal compound as claimed in claim 1, wherein the COX inhibitor is chiral.

5. The antifungal compound of Formula (I), as claimed in claim 4, wherein the chiral COX inhibitor is selected from the group consisting of ibuprofen, flurbiprofen, naproxen, and ketoprofen.

6. The antifungal compound as claimed in claim 1, wherein the COX inhibitor is achiral.

7. The antifungal compound as claimed in claim 6, wherein the achiral COX inhibitor is selected from the group consisting of niflumic acid, diflunisal, salicylic acid, and diclofenac.

8. The antifungal compound as claimed in claim 1, wherein the COX inhibitor is ibuprofen-based or flubribiprofen-based.

9-12. (canceled)

13. The antifungal compound of Formula (I), as claimed in claim 1, wherein the antifungal compound is selected from the group consisting of compounds 1-24, 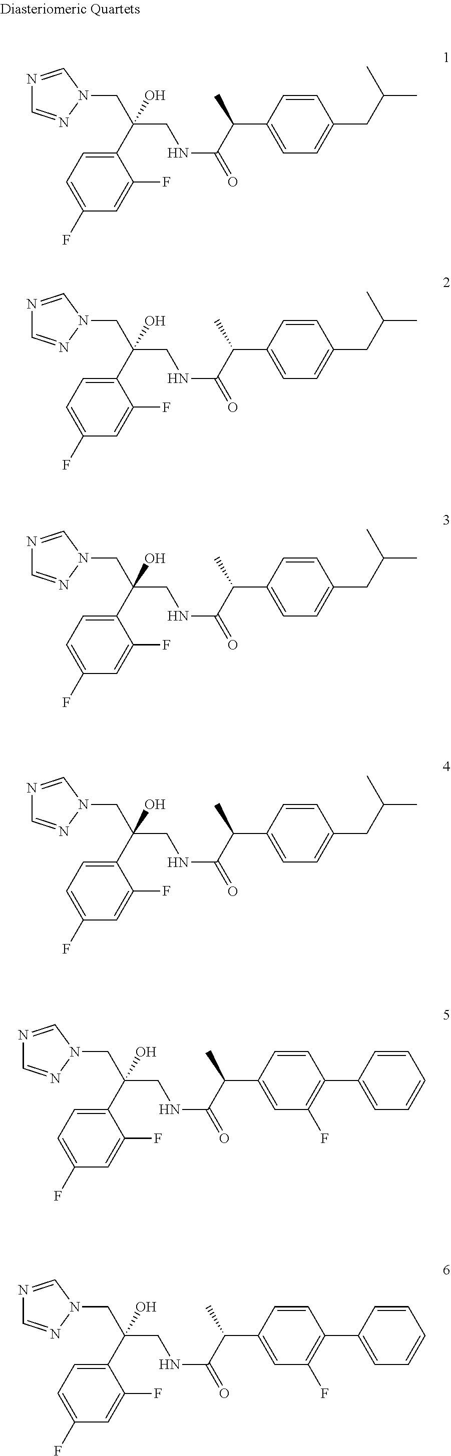 [SMILES: CC(C)Cc1ccc([C@H](C)C(=O)NC[C@](O)(Cn2cncn2)c2ccc(F)cc2F)cc1 ; CC(C)Cc1ccc([C@@H](C)C(=O)NC[C@](O)(Cn2cncn2)c2ccc(F)cc2F)cc1 ; CC(C)Cc1ccc([C@@H](C)C(=O)NC[C@@](O)(Cn2cncn2)c2ccc(F)cc2F)cc1 ; CC(C)Cc1ccc([C@H](C)C(=O)NC[C@@](O)(Cn2cncn2)c2ccc(F)cc2F)cc1 ; C[C@H](C(=O)NC[C@](O)(Cn1cncn1)c1ccc(F)cc1F)c1ccc(-c2ccccc2)c(F)c1 ; C[C@@H](C(=O)NC[C@](O)(Cn1cncn1)c1ccc(F)cc1F)c1ccc(-c2ccccc2)c(F)c1] 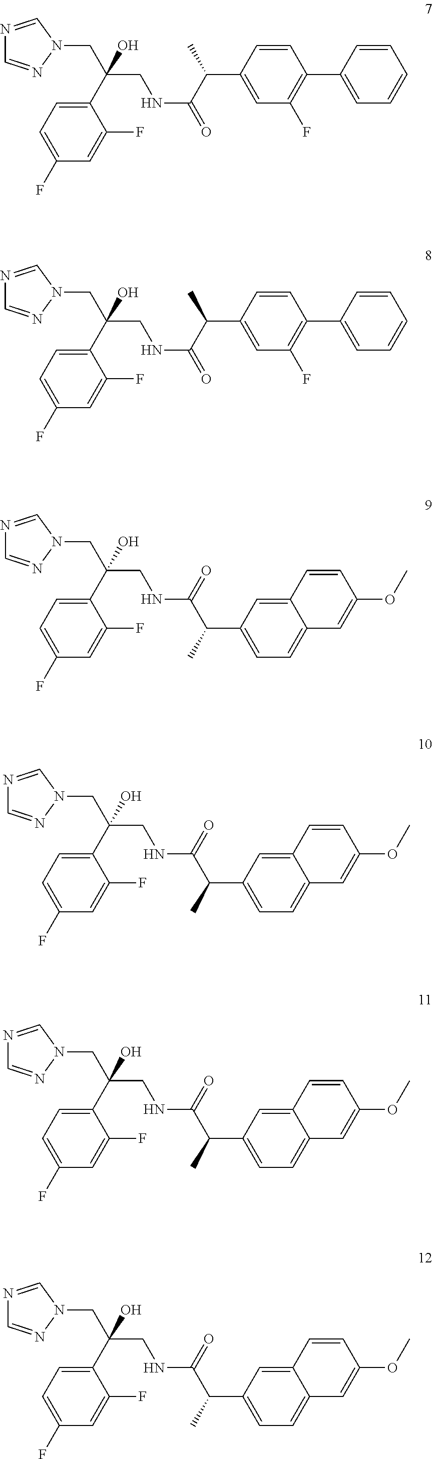 [SMILES: C[C@@H](C(=O)NC[C@@](O)(Cn1cncn1)c1ccc(F)cc1F)c1ccc(-c2ccccc2)c(F)c1 ; C[C@H](C(=O)NC[C@@](O)(Cn1cncn1)c1ccc(F)cc1F)c1ccc(-c2ccccc2)c(F)c1 ; COc1ccc2cc([C@H](C)C(=O)NC[C@](O)(Cn3cncn3)c3ccc(F)cc3F)ccc2c1 ; COc1ccc2cc([C@@H](C)C(=O)NC[C@](O)(Cn3cncn3)c3ccc(F)cc3F)ccc2c1 ; COc1ccc2cc([C@@H](C)C(=O)NC[C@@](O)(Cn3cncn3)c3ccc(F)cc3F)ccc2c1 ; COc1ccc2cc([C@H](C)C(=O)NC[C@@](O)(Cn3cncn3)c3ccc(F)cc3F)ccc2c1] 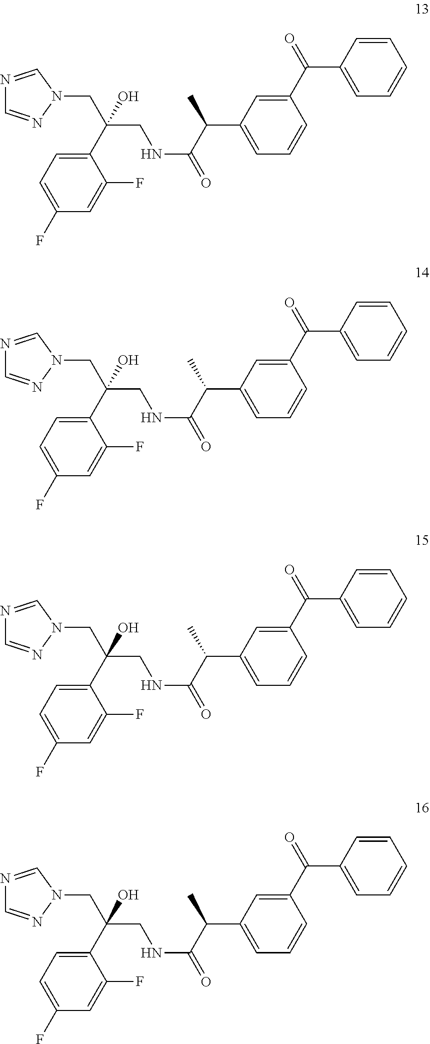 [SMILES: C[C@H](C(=O)NC[C@](O)(Cn1cncn1)c1ccc(F)cc1F)c1cccc(C(=O)c2ccccc2)c1 ; C[C@@H](C(=O)NC[C@](O)(Cn1cncn1)c1ccc(F)cc1F)c1cccc(C(=O)c2ccccc2)c1 ; C[C@@H](C(=O)NC[C@@](O)(Cn1cncn1)c1ccc(F)cc1F)c1cccc(C(=O)c2ccccc2)c1 ; C[C@H](C(=O)NC[C@@](O)(Cn1cncn1)c1ccc(F)cc1F)c1cccc(C(=O)c2ccccc2)c1] 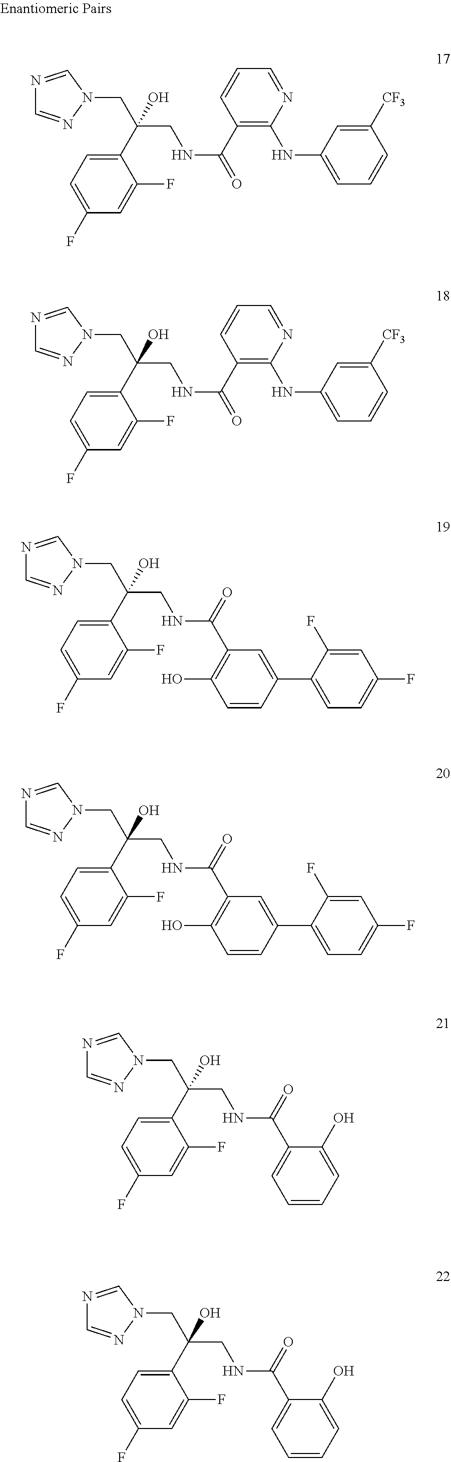 [SMILES: O=C(NC[C@](O)(Cn1cncn1)c1ccc(F)cc1F)c1cccnc1Nc1cccc(C(F)(F)F)c1 ; O=C(NC[C@@](O)(Cn1cncn1)c1ccc(F)cc1F)c1cccnc1Nc1cccc(C(F)(F)F)c1 ; O=C(NC[C@](O)(Cn1cncn1)c1ccc(F)cc1F)c1cc(-c2ccc(F)cc2F)ccc1O ; O=C(NC[C@@](O)(Cn1cncn1)c1ccc(F)cc1F)c1cc(-c2ccc(F)cc2F)ccc1O ; O=C(NC[C@](O)(Cn1cncn1)c1ccc(F)cc1F)c1ccccc1O ; O=C(NC[C@@](O)(Cn1cncn1)c1ccc(F)cc1F)c1ccccc1O] 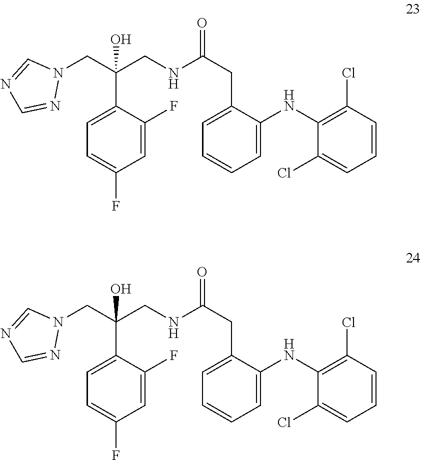 [SMILES: O=C(Cc1ccccc1Nc1c(Cl)cccc1Cl)NC[C@](O)(Cn1cncn1)c1ccc(F)cc1F ; O=C(Cc1ccccc1Nc1c(Cl)cccc1Cl)NC[C@@](O)(Cn1cncn1)c1ccc(F)cc1F]
FIG. 4
as shown in FIG. 4.

14. A method of making the antifungal compound of claim 1, said method comprising:
coupling a COX inhibitor to an azole pharmacophore.

15. (canceled)

16. The method of making the antifungal compound of claim 14, wherein the COX inhibitor is coupled to the azole pharmacophore via HATU-based amide bond formation.

17. The method of making the antifungal compound of claim 14, wherein the COX inhibitor is selected from the group consisting of ibuprofen, flurbiprofen, naproxen, ketoprofen, niflumic acid, diflunisal, salicylic acid, and diclofenac

18. The method of making the antifungal compound of claim 14, said method further comprising:
at least one of the following steps according to Scheme 1: 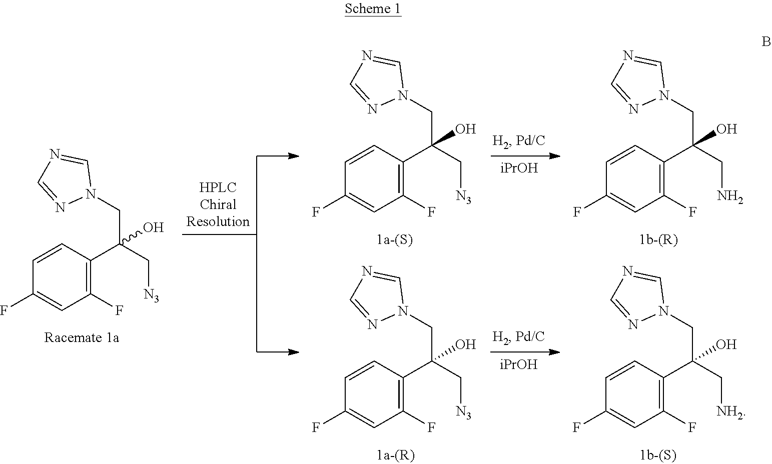 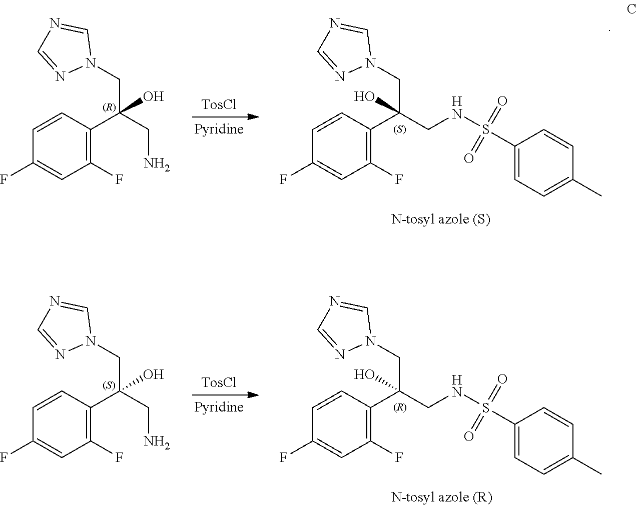
coupling a carboxyl group of a COX-inhibitor with N-tosyl azole (S) or N-tosyl azole (R) via amide bond formation.

19. The method of making the antifungal compound of claim 18, further comprising the precursor step of:
tosylating amine-functionalized azole pharmacophores 1b-(S) or 1b-(R) to obtain N-tosyl azole (S) or N-tosyl azole (R), respectively.

20. The method of making the antifungal compound of claim 18, further comprising the precursor step of:
subjecting an azide-functionalized pharmacophore of Racemate 1a to catalytic hydrogenation to obtain amine-functionalized azole pharmacophores 1b-(S) and 1b-(R).

21. The method of making the antifungal compound of claim 18, wherein the COX inhibitor is selected from the group consisting of ibuprofen, flurbiprofen, naproxen, ketoprofen, niflumic acid, diflunisal, salicylic acid, and diclofenac.

22. The method of making the antifungal compound of claim 14, wherein the step of coupling the COX inhibitor to the azole pharmacophore results in a compound selected from the group consisting of compounds 1-24, 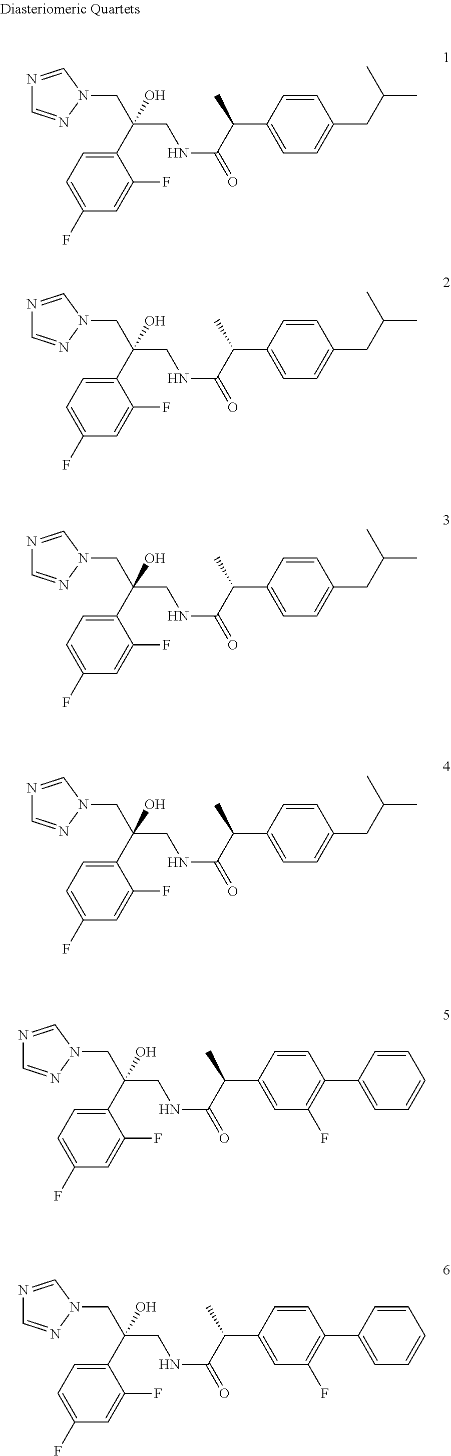 [SMILES: CC(C)Cc1ccc([C@H](C)C(=O)NC[C@](O)(Cn2cncn2)c2ccc(F)cc2F)cc1 ; CC(C)Cc1ccc([C@@H](C)C(=O)NC[C@](O)(Cn2cncn2)c2ccc(F)cc2F)cc1 ; CC(C)Cc1ccc([C@@H](C)C(=O)NC[C@@](O)(Cn2cncn2)c2ccc(F)cc2F)cc1 ; CC(C)Cc1ccc([C@H](C)C(=O)NC[C@@](O)(Cn2cncn2)c2ccc(F)cc2F)cc1 ; C[C@H](C(=O)NC[C@](O)(Cn1cncn1)c1ccc(F)cc1F)c1ccc(-c2ccccc2)c(F)c1 ; C[C@@H](C(=O)NC[C@](O)(Cn1cncn1)c1ccc(F)cc1F)c1ccc(-c2ccccc2)c(F)c1] 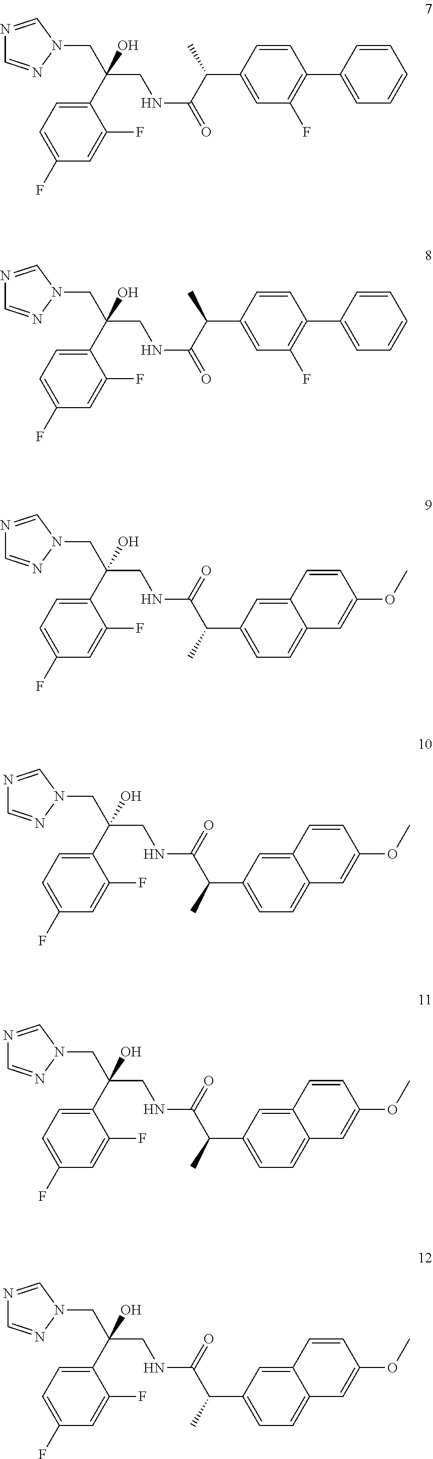 [SMILES: C[C@@H](C(=O)NC[C@@](O)(Cn1cncn1)c1ccc(F)cc1F)c1ccc(-c2ccccc2)c(F)c1 ; C[C@H](C(=O)NC[C@@](O)(Cn1cncn1)c1ccc(F)cc1F)c1ccc(-c2ccccc2)c(F)c1 ; COc1ccc2cc([C@H](C)C(=O)NC[C@](O)(Cn3cncn3)c3ccc(F)cc3F)ccc2c1 ; COc1ccc2cc([C@@H](C)C(=O)NC[C@](O)(Cn3cncn3)c3ccc(F)cc3F)ccc2c1 ; COc1ccc2cc([C@@H](C)C(=O)NC[C@@](O)(Cn3cncn3)c3ccc(F)cc3F)ccc2c1 ; COc1ccc2cc([C@H](C)C(=O)NC[C@@](O)(Cn3cncn3)c3ccc(F)cc3F)ccc2c1] 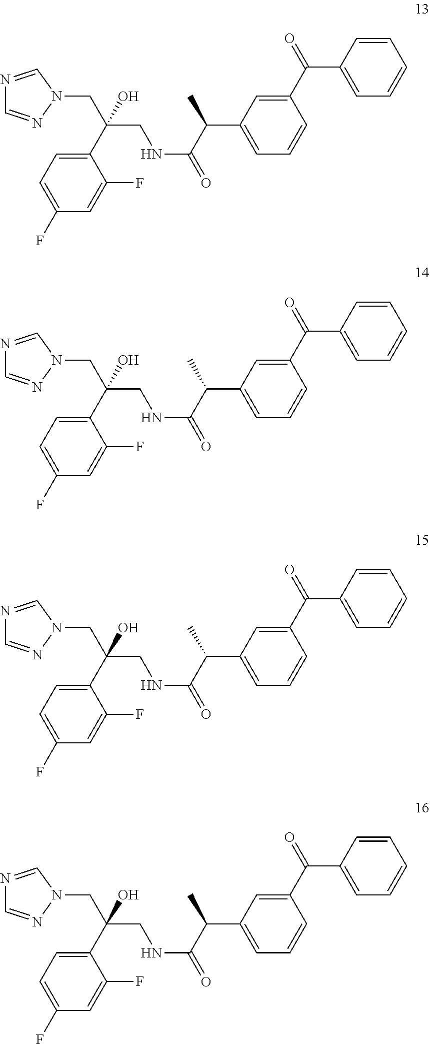 [SMILES: C[C@H](C(=O)NC[C@](O)(Cn1cncn1)c1ccc(F)cc1F)c1cccc(C(=O)c2ccccc2)c1 ; C[C@@H](C(=O)NC[C@](O)(Cn1cncn1)c1ccc(F)cc1F)c1cccc(C(=O)c2ccccc2)c1 ; C[C@@H](C(=O)NC[C@@](O)(Cn1cncn1)c1ccc(F)cc1F)c1cccc(C(=O)c2ccccc2)c1 ; C[C@H](C(=O)NC[C@@](O)(Cn1cncn1)c1ccc(F)cc1F)c1cccc(C(=O)c2ccccc2)c1] 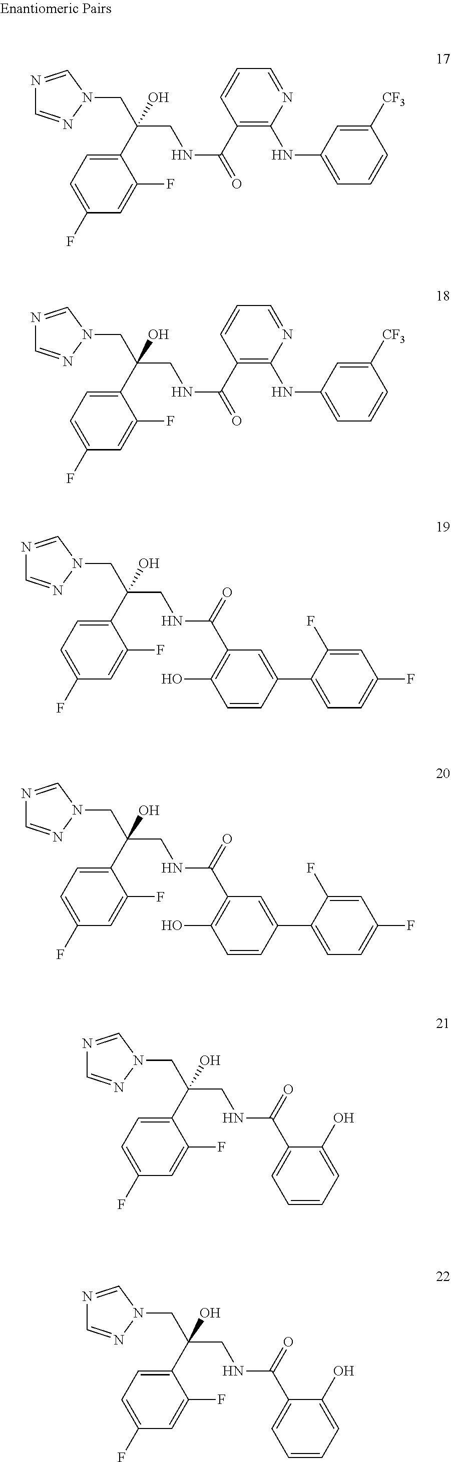 [SMILES: O=C(NC[C@](O)(Cn1cncn1)c1ccc(F)cc1F)c1cccnc1Nc1cccc(C(F)(F)F)c1 ; O=C(NC[C@@](O)(Cn1cncn1)c1ccc(F)cc1F)c1cccnc1Nc1cccc(C(F)(F)F)c1 ; O=C(NC[C@](O)(Cn1cncn1)c1ccc(F)cc1F)c1cc(-c2ccc(F)cc2F)ccc1O ; O=C(NC[C@@](O)(Cn1cncn1)c1ccc(F)cc1F)c1cc(-c2ccc(F)cc2F)ccc1O ; O=C(NC[C@](O)(Cn1cncn1)c1ccc(F)cc1F)c1ccccc1O ; O=C(NC[C@@](O)(Cn1cncn1)c1ccc(F)cc1F)c1ccccc1O] 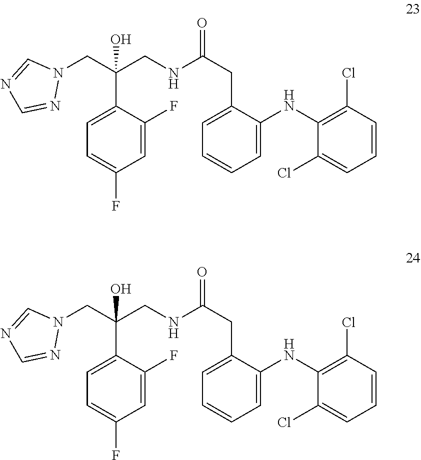 [SMILES: O=C(Cc1ccccc1Nc1c(Cl)cccc1Cl)NC[C@](O)(Cn1cncn1)c1ccc(F)cc1F ; O=C(Cc1ccccc1Nc1c(Cl)cccc1Cl)NC[C@@](O)(Cn1cncn1)c1ccc(F)cc1F]
FIG. 4 as shown in FIG. 4.

23. An antifungal composition comprising an antifungal compound of claim 1 for treatment against a fungi, the composition comprising:
an effective amount of the antifungal compound of claim 1.

24. The antifungal composition of claim 23, further comprising:
at least one additive selected from the group consisting of pharmaceutically acceptable excipient, agricultural additive, horticultural additive, or polymer additive.

25. The antifungal composition according to claim 23, wherein:
the antifungal composition is effective as a treatment or prophylactic treatment against at least one fungus of the genus Candida.

26. The antifungal composition of claim 23, wherein:
the antifungal composition is effective as a treatment or prophylactic treatment against at least one fungus selected from the group consisting of C. albicans, C. glabrata, C. parapsilosis, C. tropicalis, C. guilliermondii, C. dubliniensis, and C. auris.

27-39. (canceled)
